(19) United States
(12) Patent Application Publication
(10) Pub. No.: US 2021/0398182 A1
(43) Pub. Date: Dec. 23, 2021

(54) Information Marketplace

(71) Applicant: Michael Bilotta, Portola Hills, CA (US)

(72) Inventor: Michael Bilotta, Portola Hills, CA (US)

(21) Appl. No.: 17/466,484

(22) Filed: Sep. 3, 2021

Related U.S. Application Data
(63) Continuation of application No. 16/207,985, filed on Dec. 3, 2018, now abandoned.
Application No. 16/207,985 is a continuation-in-part of application No. 14/458,081, filed on Aug. 12, 2014, now abandoned.

(51) Int. Cl. G06Q 30/06; G06Q 30/02; G06Q 50/00

(52) U.S. Cl. CPC G06Q 30/0623; G06Q 50/01; G06Q 30/0201; G06Q 30/0282

(57) ABSTRACT

A method for enabling a user to sell or grant information filtered to align with the user's life state. This information may include at least demographic information, health information, ethnic information, social information and psychological information. The system may grant customers permission to purchase or obtain the information about the user's life. Customers are also members of the information marketplace system and may respond to user offers, or customers may offer to purchase or obtain user information. Offers are evaluated using a set of criteria before they are accepted by a user or customer; and may use the transacted information about the user's life. The method also enables the customer to rate the user according to the user's behavior, and to enable search engines, web sites, systems, databases, resources, and personnel to filter search results according to the information about the user's life. The information transaction may also allow the purchased or obtained information to be periodically or dynamically updated or renewed over time. A user's information may be transacted using a variety of transaction methods, including traditional payment options, point systems, or other means according to user and customer preferences.

### PRIORITY

This application claims the benefit of and is a Continuation of U.S. patent application Ser. No. 16/207,985, filed Dec. 3, 2018, which is a Continuation-in-Part of U.S. patent application Ser. No. 14/458,081, filed Aug. 12, 2014, both of which are incorporated in their entireties herein.

### FIELD

Embodiments of the present invention generally relate to the field of transacting (buying and selling) user's life data according and in some embodiments, specifically relate to a user's life state, used in conjunction with an information delivery system.

### BACKGROUND

Users today rely heavily on the Internet to manage information and interact with other users or organizations. Popular information portals such as Microsoft®'s MSN network and Google's iGoogle compete against one another to deliver more customized information to the Users. The delivery of information includes various tools and tactics, such as cookies and security settings, that are used by organizations to optimize the accuracy of information delivered to users and the business transactions that may follow. Facebook®, Yahoo®, Amazon®, and LinkedIn® are also examples of stored user information variants, and this stored and used information focuses on information management and commerce under the context of relationships. Accurate information is important to the users and correspondingly the organizations that provide the users goods or services. The accuracy and utility of the information may be based on the filters and preferences selected by the Users or organizations. Other organizations also provide services that transact products and services that include user information, such as eBay, eHarmony®, and Amazon®. These methods of information management, commerce, and user and organizational interaction have evolved, but there is not method of directly transacting a user's information. Current methods of obtaining user information relevant to understanding user behavior and needs focuses on indirect and intermediated channels that obscure, distort, and mutate informational accuracy and relevance by the time they get to the organization using the information. In addition to the problems with the quality and conduits, the current methods are also not the most efficient way to get information from the most accurate source to its destination.

### SUMMARY

For some embodiments, a method to enable a user to manage the offering and selling of their life state information that is based on filtered information that dynamically structures and displays the information about the user's life according to the information that is most relevant to the user at any given point in time. The information marketplace manages and transacts information regarding the user's life state, which may as also be displayed in the user's life view. The user's information marketplace may include many categorical information zones related to the user's life view, based on filters in at least areas such as demographic, ethnic, social and psychological aspects. The user may define the filters and criteria for information to be offered or sold, as part of the SpotMi Life State and Information Marketplace parameters. The user may also define which categorical areas or attributes within categorical areas that will be offered or sold and how the information that changes is managed for offering or sale. The user may specify the categories, attributes, and selling and offering parameters to change according to the user's preferences. The user information available for offering or sale is determined by user gatekeeping attributes and security, which are defined in a user's life state. (For example, the user can define security and gatekeeping attributes that will allow inclusion of all entertainment oriented attributes to automatically be available for offering or sale regardless of how those attribute values are populated or what value they are populated with). The user may also define time-phased parameters for offering and sale, which may be based on month, season, events, years, career states, geography, and other parameters. This embodiment may interface with the underlying SpotMi Information Delivery System to allow users to set gatekeeping and security values that dynamically change according to the user's Life State. The information marketplace offers user information for exchange, for sale, or via other transactional methods, to organizations or individuals that may be interested in procuring or obtaining the information. The settings or some embodiments may also include various devices, appliances, and systems. The information marketplace may integrate with a life view to allow suppliers of information to push specific information to the user's life view, through the underlying SpotMi Information Delivery System and the SpotMi Marketplace.

To this end in an exemplary embodiment, a computer-implemented method, comprising: offering for sale, granting, renting, or leasing, the information about and relevant to a user's life from a user who is a registered member of an information delivery system or information marketplace, wherein the information about and relevant to the user's life includes at least: (a) demographic information, health (b), (c) ethnic information, (d) social information, and (e) psychological information; enabling the customer to procure or obtain access to the information about and relevant to the user's life, wherein the customer is also a registered member of the information delivery system or information marketplace, and wherein the user decides which customer to sell or grant the information to in terms of information details, duration, expiration, frequency, extent, type, and volume of filtered information that is purchased or obtained by the customer; and wherein the information about and relevant to the user's life is provided or sold by the user using a client computing system associated with the information delivery system and information marketplace.

To this end in an exemplary embodiment, a computer-implemented method, comprising: offering for sale, granting, renting, or leasing, the information about and relevant to a user's life from a user who is a registered member of an information delivery system or information marketplace, wherein the information about and relevant to the user's life includes at least (a) demographic information, (b) health information, (c) ethnic information, (d) social information, and (e) psychological information; enabling the customer to procure or obtain access to the information about and relevant to the user's life, wherein the customer is also a registered member of the information delivery system or information marketplace, and wherein the user decides which customer to sell or grant the information to in terms of information details, duration, expiration, frequency, extent, type, and volume of filtered information that is purchased or obtained by the customer; and wherein the information about and relevant to the user's life is provided or sold by the user using a client computing system associated with the information delivery system, information marketplace, and life view.

In an another exemplary embodiment, comprising: providing a transaction to allow access and updates to the filtered information, wherein the filtered information may be a subset of the information available from a customer, and wherein the information transacted may be independently configurable from a life state associated with the user of the information delivery system.

In an another exemplary embodiment, comprising: providing and displaying information in a marketplace to enable customers to procure the information about the user's life. Wherein the user may prioritize, position, and display information regarding marketplace interests, bids, offers, and transactions.

In an another exemplary embodiment, comprising: establishing a data structure to enable the user to provide and offer for sale or granting the information about and relevant to the user's life, the data structure including fields related to one another, wherein each of the fields is associated with a value, and wherein the data structure includes multiple levels such that a field at a lower level is to provide more detail information and value than a corresponding field at a higher level.

In an exemplary another embodiment, comprising: at least the definition of parameters and settings involving how the information is offered, to what extent, length of time available, extent of updating the information after the transaction, and types of customers that may purchase or obtain the user's information.

In an another exemplary embodiment, comprising: enabling the user and customer to complete transactions including the user's information and updating user's information using a mobile computing system and a mobile application associated with the information delivery system and life view.

In an another exemplary embodiment, comprising: enabling the customer to rate the user based on whether the purchased or obtained information from the user is aligned with the user's behavior; and enabling the user to rate the customer based on whether the information about and relevant to the user's life is aligned with behavior of the user when the user visits a website associated with the customer or interacts or transacts with the customer or any of the customer's resources, systems, or personnel. Whereas customers may use the user ratings to assess and value information offered for sale or granting, and user may use the customer's ratings to assess and value customers that bid for or attempt to purchase or obtain the user's information.

In an another exemplary embodiment, comprising: storing the transacted information about and relevant to the user's life in a secured relational database associated with the information marketplace, information delivery system, and life view; and enabling the transactional information about and relevant to the user's life to be accessed by the customer and user when the user visits a web site of the customer or interacts with the customer, the customer's resources, systems, or personnel.

In an another exemplary embodiment, wherein customized web pages, systems, databases, and devices are presented to the user when the user interacts with the customer, and wherein the customized depictions are generated by the customer based on the information purchased or obtained about and relevant to the user's life.

In an another exemplary embodiment, wherein filtered information is presented to the user when the user interacts with the systems, resources, or personnel of the customer, and wherein such information is generated by the customer based on parameters according to the transaction, user, and based on the information about and relevant to the user's life.

In another exemplary embodiment, a computer-readable media that stores instructions, which when executed by a machine, cause the machine to perform operations comprising: purchasing or obtaining information about a user's life from a user who is a registered member of an information marketplace, wherein the information about the user's life includes at least demographic, ethnic, social and psychological information; enabling the user to set parameters to control (a) selling or granting the information about the user's life by a customer and (b) volume, frequency, parameters, and transaction types regarding filtered information to be received by the customer on from the user, wherein the customer is also a registered member of the information marketplace; and receiving the transacted information from the user, wherein the transacted information is generated by responding to offers from the user for the user's information or offering to buy or obtain information from the user; and transacting the filtered information from the user to the customer.

In an another exemplary embodiment, wherein the user and customer further sets the parameters to control (a) Type and extent of transacted information, and (b) parameters that determine how the information may be managed or used and accessed by the customer following the transaction, and (c) the transaction consideration required to complete the transaction, and (d) transactional information such as customer billing information, user information, payment information.

In an another exemplary embodiment, comprising: establishing a data structure to enable the user to provide the information about the user's life, the data structure including fields related to one another, wherein each of the fields is associated with a value representing one aspect of the user's life; automatically populating the fields of the data structure with a set of baseline values; enabling the user to manually update the baseline values or non-baseline values to more accurately reflect the user's life; enabling the user to rate the customer based on whether the filtered information pushed by the customer is aligned with the information about the user's life; and enabling the customer to rate the user based on whether the information about the user's life is aligned with behavior of the user when the user visits a website associated with the customer.

In an another exemplary embodiment, comprising: providing a marketplace for the customers, users, or other parties to purchase the information about the user's life; enabling the customer to use the information about the user's life to customize web pages, systems, and interactions of customer resources or personnel of the customer when the user interacts or transacts with a customer; enabling marketplace information to be displayed in a user life view, according to priority, positioning, and custom display settings; and enabling a user to interact with and use marketplace information in the user's life view.

In an another exemplary embodiment, wherein the marketplace is an auction-based marketplace, wherein the customized web pages, systems, databases, resources, and personnel include content that presents filtered information when that web page, system, database, resource, or personnel are associated with the filtered information.

In an another exemplary embodiment, comprising: enabling the user to cause the values of the information transaction fields of the data structure to be updated using a mobile application associated with the information marketplace; and wherein the user may access, interact with, and modify settings for the information marketplace according to the embodiments herein.

In an another exemplary embodiment, wherein the mobile application is configured to enable the user to control and manage transactions and updates that relate to the information marketplace.

In an exemplary embodiment, a system, comprising: information delivery and marketplace servers connected to a network, the information delivery and marketplace servers configured to: (a) enable a user to sell or grant information about a user's life using a client computing system coupled with the information marketplace server, wherein the information about the user's life includes at least demographic, health, ethnic, social and psychological information, and wherein the information about the user's life is stored in a database coupled with the information marketplace server, the client computing system connected to the network; b) enable the user and customer to set parameters that control transactions including the information about the user's life by volume, static or dynamic nature, type, or duration of information to be sold or granted to the customer from the user, wherein the information received from the user is to be aligned with the information about the user's life, and wherein the customer is associated with a customer server connected to the network; (c) enable the customer to rate the user based on whether behavior of the user when the user visits a web site, system, database, resource, or personnel of the customer is aligned with the information about the user's life; and (d) enable the user to rate the customer based on whether the information received from the customer is aligned with the information about the user's life.

In an another exemplary embodiment, wherein the information marketplace server is further configured to (a) provide a marketplace to enable the customer to purchase the information about the user's life and (b) update a life view to present the information received from the customer to the user according to priorities, positioning settings, and other user defined settings.

In an another exemplary embodiment, wherein the user and the customer are registered with the information marketplace server, and wherein the information marketplace server is further configured to (a) receive updated information about the user's life transactions from the user and customer via a mobile computing device, and (b) enable the customer to access the information about the user's life to customize the customer's websites, systems, databases, resources, or personnel to integrate the information about the user's life, and enable the customer to receive user information transaction updates from the user's life state, and (d) enable the user to access, update, and interact with a user life view via a mobile computing device.

In another exemplary embodiment, further comprising: providing a transaction to allow access and updates to the filtered information, wherein the filtered information may be a subset of the information available from a customer, and wherein the information transacted may be independently configurable from a life state associated with the user of the information delivery system.

In another exemplary embodiment, further comprising: providing and displaying information in a marketplace to enable customers to procure the information about the user's life. Wherein the user may prioritize, position, and display information regarding marketplace interests, bids, offers, and transactions.

In another exemplary embodiment, further comprising: establishing a data structure to enable the user to provide and offer for sale or granting the information about and relevant to the user's life, the data structure including fields related to one another, wherein each of the fields is associated with a value, and wherein the data structure includes multiple levels such that a field at a lower level is to provide more detail information and value than a corresponding field at a higher level.

In another exemplary embodiment, further comprising: at least the definition of parameters and settings involving how the information is offered, to what extent, length of time available, extent of updating the information after the transaction, and types of customers that may purchase or obtain the user's information.

In another exemplary embodiment, further comprising: enabling the user and customer to complete transactions including the user's information and updating user's information using a mobile computing system and a mobile application associated with the information delivery system.

In another exemplary embodiment, further comprising: enabling the customer to rate the user based on whether the purchased or obtained information from the user is aligned with the user's behavior; and enabling the user to rate the customer based on whether the customer products, services, offerings, or information about and relevant to the user's life is aligned with the behavior of the user when the user visits a web site associated with the customer or interacts or transacts with the customer or any of the customer's resources, systems, or personnel. Whereas customers may use the user ratings to assess and value information offered for sale or granting, and user may use the customer's ratings to assess and value customers that bid for or attempt to purchase or obtain the user's information.

In another exemplary embodiment, further comprising: storing the transacted information about and relevant to the user's life in a secured relational database associated with the information marketplace and information delivery system; and enabling the transactional information about and relevant to the user's life to be accessed by the customer and user when the user visits a web site of the customer or interacts with the customer, the customer's resources, systems, or personnel.

In another exemplary embodiment, wherein customized web pages, systems, databases, and devices are presented to the user when the user interacts with the customer, and wherein the customized depictions are generated by the customer based on the information purchased or obtained about and relevant to the user's life.

In another exemplary embodiment, wherein filtered information is presented to the user when the user interacts with the systems, resources, or personnel of the customer, and wherein such information is generated by the customer based on parameters according to the transaction, user, and based on the information about and relevant to the user's life.

In an exemplary embodiment, a computer-readable media that stores instructions, which when executed by a machine, cause the machine to perform operations comprising: purchasing or obtaining information about a user's life from a user who is a registered member of an information marketplace, wherein the information about the user's life includes at least demographic, health, ethnic, social and psychological information; enabling the user to set parameters to control (a) selling or granting the information about the user's life by a customer and (b) volume, frequency, parameters, and transaction types regarding filtered information to be received by the customer on from the user, wherein the customer is also a registered member of the information marketplace; receiving the transacted information from the user, wherein the transacted information is generated by responding to offers from the user for the user's information or offering to buy or obtain information from the user; and transacting the filtered information from the user to the customer.

In another exemplary embodiment, wherein the user and customer further sets the parameters to control (a) Type and extent of transacted information, (b) parameters that determine how the information may be managed or used by the customer following the transaction, (c) the transaction consideration required to complete the transaction, and (d) transactional information such as customer billing information, user information, payment information.

In another exemplary embodiment, further comprising: establishing a data structure to enable the user to provide the information about the user's life, the data structure including fields related to one another, wherein each of the fields is associated with a value representing one aspect of the user's life; automatically populating the fields of the data structure with a set of baseline values; enabling the user to manually update the baseline values or non-baseline values to more accurately reflect the user's life; enabling the user to rate the customer based on whether the filtered information pushed by the customer is aligned with the information about the user's life; and enabling the customer to rate the user based on whether the information about the user's life is aligned with behavior of the user when the user visits a website associated with the customer.

In another exemplary embodiment, further comprising: providing a marketplace for the customers, users, or other parties to purchase or obtain the information about the user's life; and enabling the customer to use the information about the user's life to customize web pages, systems, and interactions of customer resources or personnel of the customer when the user interacts or transacts with a customer; and enabling marketplace information to be displayed in a user life view, according to priority, positioning, and custom display settings; and enabling a user to interact with and use marketplace information in the user's life view.

In another exemplary embodiment, wherein the marketplace is an auction-based marketplace, wherein the customized web pages, systems, databases, resources, and personnel include content that presents filtered information when that web page, system, database, resource, or personnel are associated with the filtered information.

In another exemplary embodiment, further comprising: enabling the user to cause the values of the information transaction fields of the data structure to be updated using a mobile application associated with the information marketplace; and wherein the user or customer may access, interact with, and modify settings for the information marketplace according to the embodiments herein.

In another exemplary embodiment, wherein the mobile application is configured to enable the user or customer to control and manage transactions and updates that relate to the information marketplace.

In an exemplary embodiment, a system, comprising: information delivery and marketplace servers connected to a network, the information delivery and marketplace servers configured to: (a) enable a user to sell or grant information about a user's life using a client computing system coupled with the information marketplace server, wherein the information about the user's life includes at least demographic, health, ethnic, social and psychological information, and wherein the information about the user's life is stored in a database coupled with the information marketplace server, the client computing system connected to the network; (b) enable the user and customer to set parameters that control transactions including the information about the user's life by volume, static or dynamic nature, type, or duration of information to be sold or granted to the customer from the user, wherein the information received from the user is to be aligned with the information provided by the customer about the user's life, and wherein the customer may be associated with a customer server connected to the network; (c) enable the customer to rate the user based on whether behavior of the user when the user visits a web site, system, database, resource, or personnel of the customer is aligned with the information obtained or purchased by the customer about the user's life; and (d) enable the user to rate the customer based on whether the information received from the customer is aligned with the information about the user's life.

In another exemplary embodiment, wherein the information marketplace server is further configured to (a) provide a marketplace to enable the customer to purchase or obtain the information about the user's life and (b) update a life view to reflect information about information offered for sale or granting by the user for customers or prospective customers, and (c) update a life view to present the information received from the customer to the user according to priorities, positioning settings, and other user defined settings.

In another exemplary embodiment, wherein the user and the customer are registered with the information marketplace server, and wherein the information marketplace server is further configured to: (a) receive updated information about the user's life transactions or updates from the user and customer via a mobile computing device; (b) enable the customer to access the information about the user's life to customize the customer's websites, systems, databases, resources, or personnel to integrate the information about the user's life, and enable the customer to receive user information transaction updates from the user's life state; and (c) enable the user to access, update, and interact with a user life view via a mobile computing device.

### BRIEF DESCRIPTION OF THE DRAWINGS

The multiple drawings refer to the embodiments of the invention. While embodiments of the invention described herein is subject to various modifications and alternative forms, specific embodiments thereof have been shown by way of example in the drawings and will herein be described in detail.

FIG. 1 illustrates one example of a computing device that may be used, in accordance with some embodiments.

FIG. 2 illustrates an example of a network that may be used to pull filtered information, in accordance with some embodiments.

FIG. 3A illustrates an example structure of the information marketplace, in accordance with some embodiments.

FIG. 3B illustrates an example of a collection life states and information marketplace attributes, in accordance with some embodiments.

FIG. 3C illustrates an example of to offers, bids, categories, and attributes, for the information marketplace, in accordance with some embodiments.

FIG. 3D illustrates an example diagram of a group of customers, in accordance with some embodiments.

FIG. 3E illustrates an example diagram of the information marketplace, in accordance with some embodiments.

FIG. 3F illustrates an example diagram of information marketplace, attributes, and the transactions, in accordance with some embodiments.

FIG. 4 illustrates example applications that may be used to enable the users to offer and sell information to users, organizations, or other third parties, in accordance with some embodiments.

FIG. 5 illustrates an example of a network with an information delivery server, in accordance with some embodiments.

FIG. 6 illustrates an example flow diagram of a process that may be performed by an information delivery and marketplace system to allow offering, selling, and transacting the user life state information, in accordance with some embodiments.

FIG. 7 illustrates an example flow diagram of a process that may be performed by an information delivery system and marketplace to allow the users and customers to perform information transactions, in accordance with some embodiments.

FIG. 8 illustrates an example flow diagram of a process that may be performed on an information delivery and marketplace server to enable tracking of a user's behavior on partner websites view, in accordance with some embodiments.

FIG. 9 illustrates an example flow diagram of a process used to rate a user's behavior as compared to the life state information obtained or procured by a customer or 3^{rd} party, in accordance with some embodiments.

FIG. 10 illustrates an example flow diagram of a process that may be used to offer, sell, buy, and use obtained or procured information in the information marketplace.

### DETAILED DESCRIPTION

For some embodiments, a method for selling, procuring, and obtaining a user's information is disclosed. The method comprises enabling a user to register and become a member of an information marketplace that sells, procures, and obtains user information, which may be based on the user's life state. Information may be offered for sale or granting based on user life state, security attributes, gatekeeping attributes, and marketplace parameters. The user accesses or defines information about the user's life which is also known as a life state. The user's life state may span many areas including at least demographic information, health information, ethnic information, social information, behavioral information, and psychological information. The user may be the owner of such information and may grant third parties permission to obtain or procure the user's information. The third parties may also be members of the information delivery system

In the following description, numerous specific details are set forth, such as examples of specific data signals, components, connections, displays, configurations, etc. in order to provide a thorough understanding of the present invention. It will be apparent, however, to one skilled in the art that the present invention may be practiced without these specific details. In other instances, well known components or methods have not been described in detail but rather in block diagrams in order to avoid unnecessarily obscuring the present invention. Thus, the specific details set forth are merely exemplary. The specific details may be varied from and still be contemplated to be within the spirit and scope of the present invention.

The present invention also relates to apparatus for performing the operations herein. This apparatus may be specifically constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, and each coupled with a computing system bus. Portions of any modules or components described herein may be implemented in lines of code in software, configured logic gates in software, or a combination of both, and the portions implemented in software are tangibly stored on a computer readable storage medium.

The algorithms and displays presented herein are not inherently related to any particular computer or other apparatus. Various general purpose systems may be used with programs in accordance with the teachings herein, or it may prove convenient to construct more specialized apparatus to perform the required method blocks. The required structure for a variety of these systems will appear from the description below.

In the following description of exemplary embodiments, reference is made to the accompanying drawings that form a part hereof, and in which it is shown by way of illustration specific embodiments in which the invention can be practiced. It is to be understood that other embodiments can be used and structural changes can be made without departing from the scope of the embodiments of this invention. As used herein, the terms “couple,” “connect,” and “attach” are interchangeable and include various forms of connecting one part to another either directly or indirectly. Also, it should be appreciated that one or more structural features described in one embodiment could be implemented in a different embodiment, even if not specifically mentioned as being a feature thereof.

### Computer System

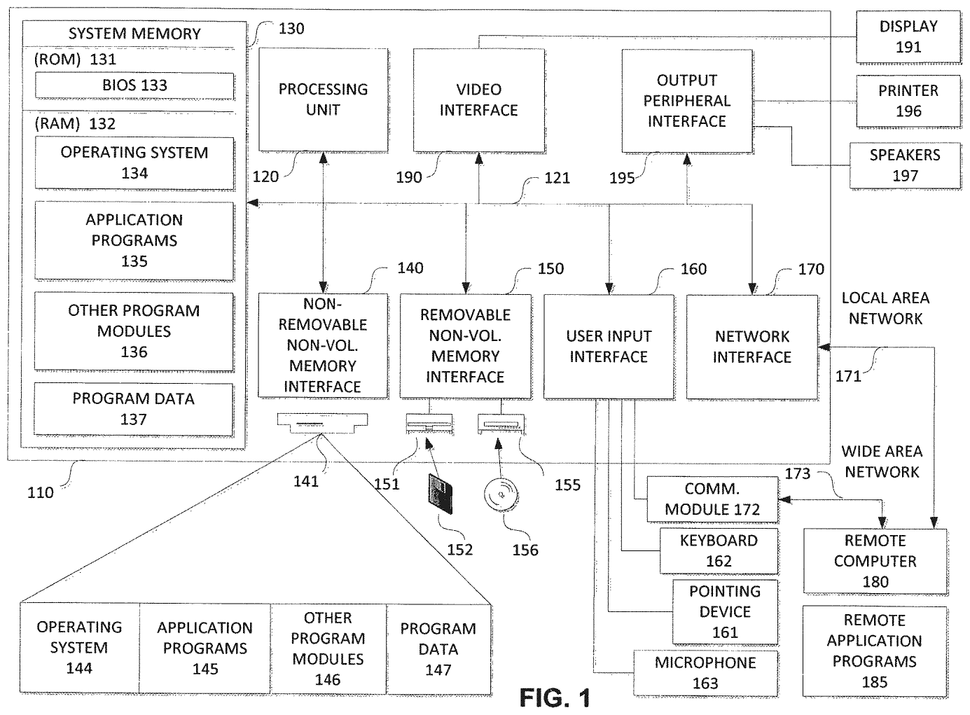

FIG. 1 illustrates a block diagram of an example computing system that may use an embodiment of one or more of the software applications discussed herein, in accordance with some example embodiments. The computing system environment 100 is only one example of a suitable computing environment, such as a client device, and is not intended to suggest any limitation as to the scope of use or functionality of the design. Neither should the computing environment 100 be interpreted as having any dependency or requirement relating to any one or combination of components illustrated in the exemplary operating environment 100. Embodiments of the invention may be implemented on any type of computer, device, apparatus, or even machine that make use of the computer environment 100. For example, the device may be an entertainment device that allows the user to offer the user's entertainment history, behavior, or preferences for sale.

The design is operational with numerous other general purpose or special purpose computing system environments or configurations. Examples of well-known computing systems, environments, and/or configurations that may be suitable for use with the design include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, automotive and informational appliances, and the like.

The design may be described in the general context of computing device executable instructions, such as program modules, being executed by a computer. Generally, the program modules include routines, programs, objects, components, data structures, etc., that perform particular tasks or implement particular abstract data types. Those skilled in the art can implement the description and/or figures herein as computer-executable instructions, which can be embodied on any form of computing machine readable media discussed below.

The design may also be practiced in distributed computing environments where tasks are performed by remote processing devices that are linked through a communications network. In a distributed computing environment, program modules may be located in both local and remote computer storage media including memory storage devices.

With reference to FIG. 1, an exemplary computing type system for implementing the design includes a general-purpose computing device in the form of a computing device 110. Components of computing device 110 may include, but are not limited to, a processing unit 120 having one or more processing cores, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) locale bus, and Peripheral Component Interconnect (PCI) bus.

Computing device 110 typically includes a variety of computing machine readable media. Computing machine readable media can be any available media that can be accessed by computing device 110 and includes both volatile and nonvolatile media, removable and non-removable media. By way of example, and not limitation, computing machine readable mediums uses include storage of information, such as computer readable instructions, data structures, program modules or other data. Computer storage mediums include, but are not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 100. Communication media typically embodies computer readable instructions, data structures, program modules, or other transport mechanism and includes any information delivery media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computing device 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computing device 110 may also include other removable/non-removable volatile/nonvolatile computer storage media. By way of example only, FIG. 1 illustrates a hard disk drive 141 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, USB drives and devices, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computing device 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies.

A user may enter commands and information into the computing device 110 through input devices such as a keyboard 162, a microphone 163, and a pointing device 161, such as a mouse, trackball or touch pad. Other input devices (not shown) may include a joystick, game pad, satellite dish, touchscreen, gesture based input, virtual reality head-mounted display, scanner, or the like. These and other input devices are often connected to the processing unit 120 through a user input interface 160 that is coupled to the system bus, but they may be connected by other interface and bus structures, such as a parallel port, game port or a universal serial bus (USB). A monitor or display 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computing device 110 may operate in a networked environment using logical connections to one or more remote computers, such as a remote computer 180. The remote computer 180 may be a personal computer, a hand-held device, a wearable device, a server, a router, a network PC, a peer device or other common network node, and typically includes many or all of the elements described above relative to the computing device 110. The logical connections depicted in FIG. 1 include a local area network (LAN) 171 and a wide area network (WAN) 173, but may also include other networks. Such networking environments are commonplace in offices, enterprise-wide computer networks, intranets, extranets, and the Internet. A browser application may be resident on the computing device and stored in the memory.

When used in a LAN networking environment, the computing device 110 is connected to the LAN 171 through a network interface or adapter 170. When used in a WAN networking environment, the computing device 110 typically includes a communication module 172 or other means for establishing communications over the WAN 173, such as the Internet. The communication module 172 may be a modem used for wired, wireless communication or both. The communication module 172 may be internal or external, may be connected to the system bus 121 via the user-input interface 160, or other appropriate mechanism. In a networked environment, program modules depicted relative to the computing device 110, or portions thereof, may be stored in the remote memory storage device. By way of example, and not limitation, FIG. 1 illustrates remote application programs 185 as residing on remote computer 180. It will be appreciated that the network connections shown are exemplary and other means of establishing a communications link between the computers may be used.

It should be noted that the present design can be carried out on a computing system such as that described with respect to FIG. 1. However, the present design can be carried out on a server, a computer devoted to message handling, on a distributed system in which different portions of the present design are carried out on different parts of the distributed computing system, or on any type of device, appliance, or apparatus where the design may be carried out.

Another device that may be coupled to bus 111 is a power supply such as a battery and Alternating Current adapter circuit. As discussed above, the DC power supply may be a battery, a fuel cell, or similar DC power source that needs to be recharged on a periodic basis. For wireless communication, the communication module 172 may employ a Wireless Application Protocol or protocol that is similar in function to establish a wireless communication channel. The communication module 172 may implement a wireless networking standard such as Institute of Electrical and Electronics Engineers (IEEE) 802.11 standard, IEEE std. 802.11-1999, published by IEEE in 1999. Other wireless communication methods that may also be used may include Bluetooth, Radio Frequency, etc.

While other systems may use, in an independent manner, various components that may be used in the design, a comprehensive, integrated system that addresses the multiple advertising system points of vulnerability described herein does not exist. Examples of mobile computing devices may be a laptop computer, a cell phone, a personal digital assistant, or other similar device with on board processing power and wireless communications ability that is powered by a Direct Current (DC) power source that supplies DC voltage to the mobile device and that is solely within the mobile computing device and needs to be recharged on a periodic basis, such as a fuel cell or a battery.

It is possible for some embodiments of the invention to have a computing environment 100 that entails memory and display functions without a processing function. For example, a third party may provide customized information to a user, which is relayed to a device for display only with no data manipulation necessary.

### Network Environment

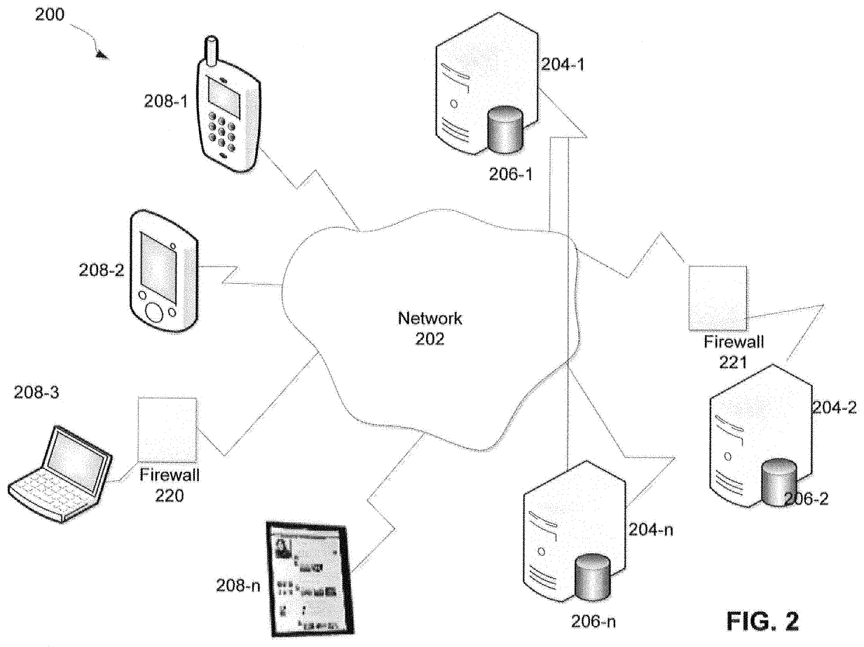

FIG. 2 illustrates a network environment 200 in which the techniques described may be applied, in accordance with some example embodiments. The network environment 200 has a network 202 that connects server computing systems 204-1 through 204-n, and at least one or more client computing systems 208-1. As shown, there may be many server computing systems 204-1 through 204-n and many client computing systems 208-1 through 208-n connected to each other via a network 202, which may be, for example, the Internet. Note, that alternatively the network 202 might be or include one or more of: an optical network, the Internet, a Local Area Network (LAN), Wide Area Network (WAN), satellite link, fiber network, cable network, or a combination of these and/or others. It is to be further appreciated that the use of the terms client computing system and server computing system is for clarity in specifying who initiates a communication (the client computing system) and who responds (the server computing system). No hierarchy is implied unless explicitly stated. Both functions may be in a single communicating device, in which case the client-server and server-client relationship may be viewed as peer-to-peer. Thus, if two systems such as the client computing system 208-1 and the server computing system 204-1 can both initiate and respond to communications, their communication may be viewed as peer-to-peer. Likewise, communications between the client computing systems 204-1, 204-2 and the server computing systems 208-1, 208-2 may be viewed as peer-to-peer if each such communicating device is capable of initiation and response to communication. One or more of the server computing systems 204-1 to 204-n may be associated with a database such as, for example, the databases 206-1 to 206-n. A firewall such as, for example, the firewall 220 between a client computing system 208-3 and the network 202, may be used to protect data integrity. Another firewall example is the firewall 221 positioned between the server 204-2 and the network 202. Although not shown, other devices (e.g., proxy servers, etc.) may also be connected to the network 202.

FIG. 2 also illustrates a block diagram of an embodiment of a server computing system to display information, such as a web page, etc. A user via a browser from the client computing system 208-1 may interact with the web page, and then supply input to the query/fields and/or service presented by a user interface of the application. The web page may be served by a web server computing system 204-1 on any Hypertext Markup Language (HTML) or Wireless Access Protocol (WAP) enabled client computing system 208-1 or any equivalent or derivative thereof. For example, the client mobile computing system 208-1 may be a smart phone, a touch pad, a laptop, a notebook, etc. The client computing system 208-1 may host a browser to interact with the server computing system 204-1. Each application, widget, plug-in, etc. has a code scripted to perform the functions that the software component is coded to carry out such as presenting fields and icons to take details of desired information. The information marketplace applications may be hosted on the server computing system 204-1 and served to the browser of the client computing system 208-1. The applications may serve as pages that allow entry of details and further pages that allow entry of more details.

Any application and other scripted code components may be stored on a computing machine readable medium which, when executed on the server causes the server to perform those functions. In an embodiment, the software used to facilitate the functions and processes described herein can be embodied onto a computing machine readable medium such as computer readable medium. As discussed above a computing machine readable medium includes any mechanism that provides (e.g., stores) information in a form readable by a machine (e.g., a computer). For example, a computing machine readable medium includes read only memory (ROM); random access memory (RAM); magnetic disk storage media; optical storage media; flash memory devices; DVD' s, EPROMs, EEPROMs, FLASH, magnetic or optical cards, or any type of media suitable for storing electronic instructions. The information representing the apparatuses and/or methods stored on the computing machine readable medium may be used in the process of creating the apparatuses and/or methods described herein.

Some portions of the detailed descriptions which follow are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. These algorithms may be written in a number of different software programming languages such as C, C++, SQL, Java, or other variants of such or similar languages. Also, an algorithm may be implemented with lines of code in software, configured logic gates in software, or a combination of both. In an embodiment, the logic consists of electronic circuits that follow the rules of Boolean Logic, software that contain patterns of instructions, or any combination of both.

It should be noted that the configuration of the diagram illustrated in FIG. 2 is for illustrative purposes only, and the actual configurations may vary. There might be different configurations of servers, firewalls, clients, and devices. For example, the devices may be anything from iPads, to Autos, to TV devices, and so on.

It should be borne in mind, however, that all of these and similar terms are to be associated with the appropriate physical quantities and are merely convenient labels applied to these quantities. Unless specifically stated otherwise as apparent from the above discussions, it is appreciated that throughout the description, discussions utilizing terms such as “processing” or “computing” or “calculating” or “determining” or “displaying” or the like, refer to the action and processes of a computing system, or similar electronic computing device, that manipulates and transforms data represented as physical (electronic) quantities within the computing system's registers and memories into other data similarly represented as physical quantities within the computing system memories or registers, or other such information storage, transmission or display devices.

### Overview

As the Internet and Advertising industry continue to evolve into a more intelligent network of usable information, there remains a significant gap between what the users want to share, what information third parties can actually obtain, and the quality of user information shared with third parties. Current methods of sharing information have many limitations that embodiments of the present invention address. Following are some of the factors regarding the limitations of the current information gathering and one-to-one marketing techniques using the internet: (a) users may not accurately understand when they are sharing information with another party; (b) users may not accurately understand what information they are sharing with another party; (c) even if the mechanics behind information sharing are known to the user, the quality and accuracy of the information shared is low and the information is usually abstract and not specific; (d) the information may be unclear, esoteric and not specific to a user's behavior, preferences, intentions, or what information the third parties seek; (e) because of a User's privacy rights, the information shared may violate some of the users privacy rights, whether known or not by failing to accurately take into consideration factors hidden or limited by the privacy rights; (f) because of privacy rights, the information obtained may be invasive to a User's privacy rights, as the current method of managing privacy rights is flawed; (g) since the number of organizations and user information is theoretically infinite, there are insufficient mechanisms that cause third parties that obtain user information to use a method of continuous improvement and focus to yield more accurately filtered information according to the user's privacy rights and preferences; (h) due to informational collection and aggregation methods and instruments, there are insufficient methods or instruments that allow information shared by a user to be visible or accurately related to a specific timeframe that relates to the user's behavior or preferences; (i) there are insufficient methods or instruments that allow the impact or strength in correlation of the user's behavior and/or preferences, as related to the factors important to a third party (driven by the User's true specification of such impact or strength of a preference or behavior); and (j) there are no feedback mechanisms that convey to the third party the relevance and accuracy of the information obtained by the third party, as compared to the User's actual behavior.

Embodiments of the invention fully utilize the information about a user's life to enable offering for sale and the procuring of filtered and customized information, and allowing the user's information to be depicted in dynamically changing areas within a life view. The information about the user's life is referred to as a life state, while how the information is used, displayed, ordered, and filtered is referred to as the life view, and how the information is offered for sale, procured, and transacted is referred to as the information marketplace. The life state may consist of many user information categories, which are dynamically offered for sale, procured, and transacted in the information marketplace. A user may grant third parties access to procure or obtain information from user's life state that is offered for sale by setting attribute, security, and gatekeeping features. As will be described, embodiments of the present invention may include an information marketplace that offers filtered information from the users for sale or use by third parties, other individuals, or other users. The information delivery system enables third parties to have access to the users' life state information, as allowed in transactions between the users and the third parties. Each user is associated with a unique life state. The information marketplace is used as an online marketplace for a user's information to be offered for sale or otherwise allowing access and use of the information by third parties. Embodiments of the invention also include an architecture that includes a centralized server computer system connected to the Internet and configured to transact user information between users and third parties. The centralized server computer system is referred to herein as an information marketplace server. The information marketplace server may be configured to provide the users a life based informational marketplace to enable the users to provide the values for the attributes and sub-attributes associated with their life states for sale or to be obtained by third parties. The information marketplace server is coupled with a life state database configured to store and secure the values for the attributes and the sub-attributes. The information marketplace server is configured to provide the users an interface to configure their own marketplace views, transaction areas and parameters, which are referred to as the users' marketplace. The users may use their marketplace to manage their offers, transactions, and view the marketplace related information. The information marketplace server may also be configured to enable the users and the third parties to rate the information transacted between one another. Replication may be involved to make access, delivery and update operations by the information marketplace server more efficient. The transactions may include the users getting compensated for allowing the third parties to access the users' life state, the third parties bidding against one another to earn the right to access the users' life states, etc. In addition, the information delivery server may be configured to enable the user's life state to be used when the user visits a website of a third party. This enables the third party to provide web pages that are customized according to the user's life state.

### Life State

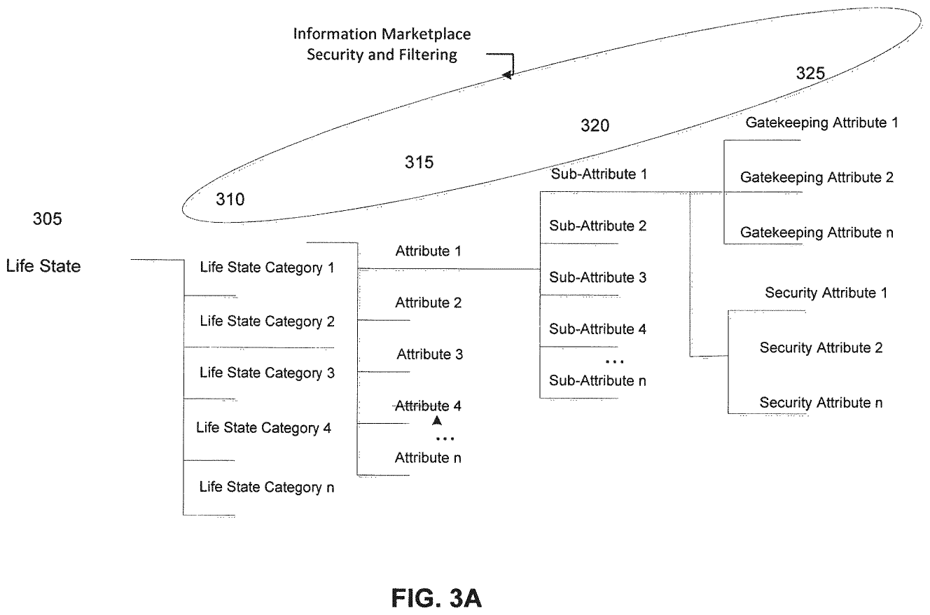

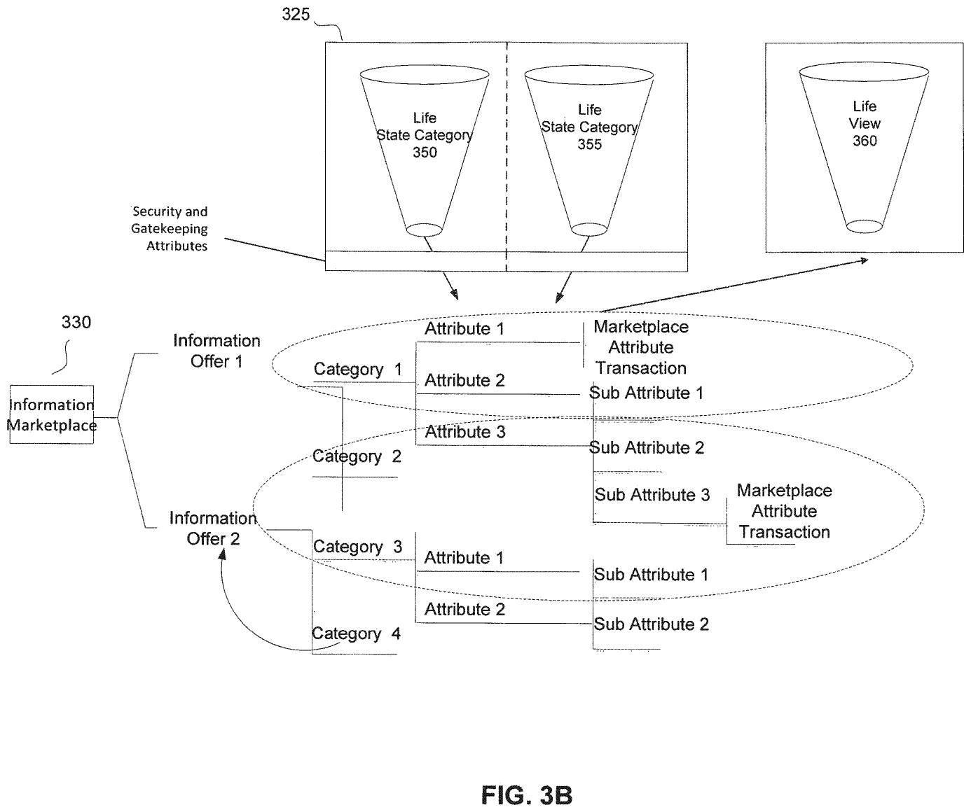

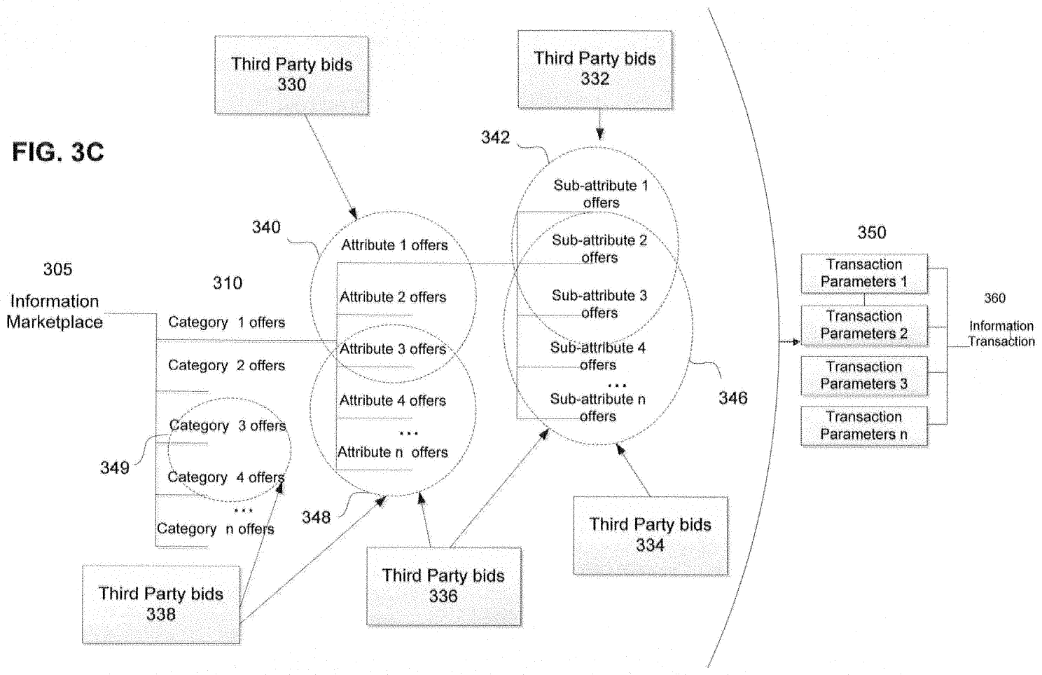

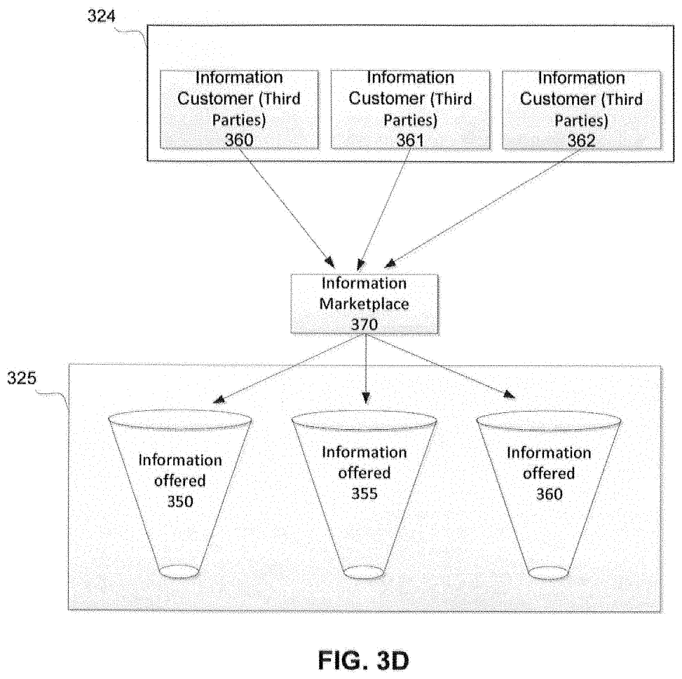

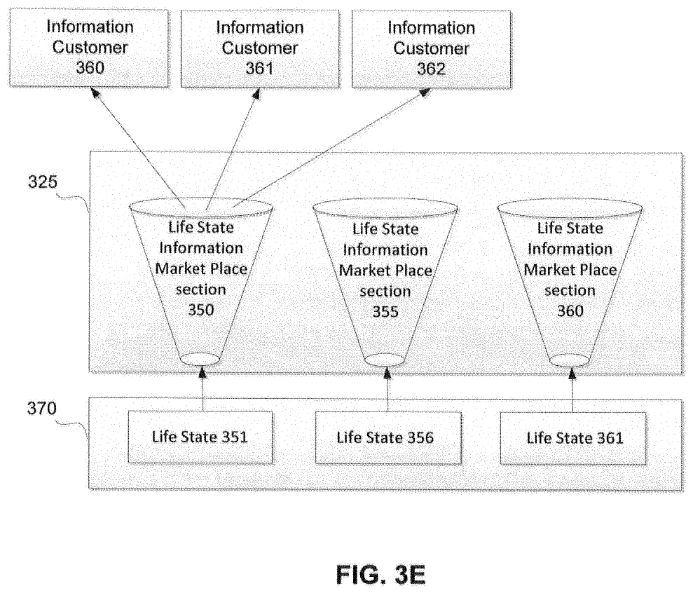

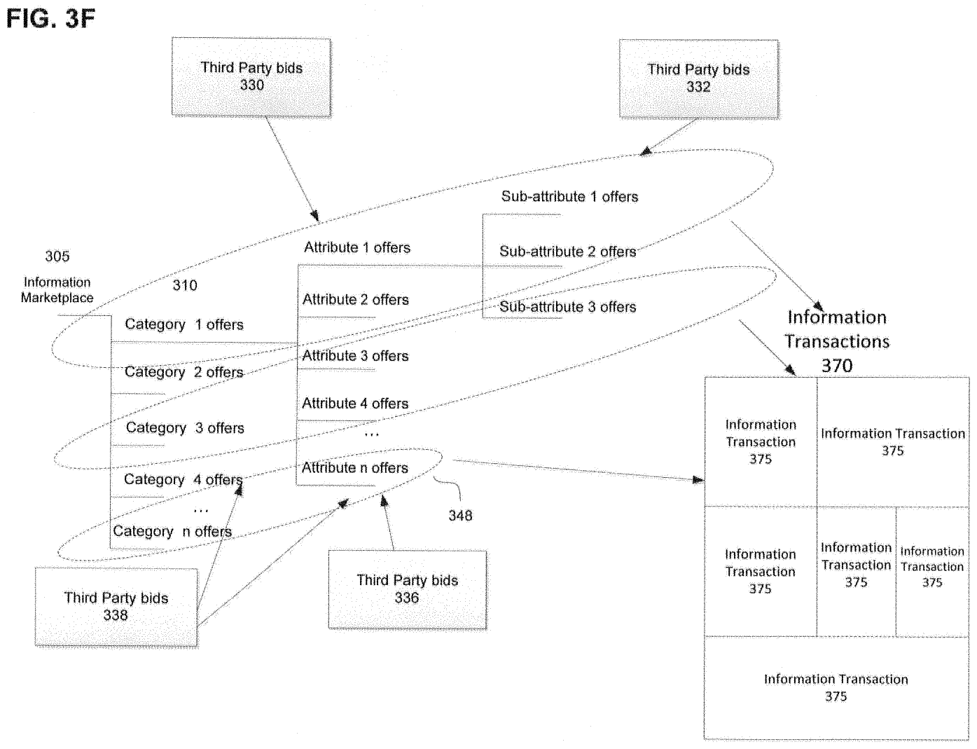

FIG. 3A illustrates an example structure of an information marketplace, in accordance with some embodiments. An information marketplace may be associated with many sellers or users, buyers or users, information category or attribute sections, custom category or attribute sections, or dynamic category or attribute sections. Information, information categories, attributes, sub-attributes, may be offered for sale or granting by the information owner or user. Each category section may consist of many attributes. Each attribute may consist of many sub-attributes. Each sub-attribute may also consist of many additional lower level sub-attributes. Together or individually, the category sections, the attributes, sub-attributes, and their corresponding values may comprise a transaction between the information owner or user and a buyer. The category sections, attributes, sub-attributes, and values may be related to one another in a top-down relationship and/or laterally-enhancing the interpretations of each attribute value. The category sections, the attributes, sub-attributes, and values may dynamically change over time or may remain unchanged for longer periods of time. The information's expiration may be factored into the transaction price and value. In some embodiments, category sections, attributes, and sub-attributes may also vary based on standards and changes by industry authorities, since industry authorities play a major role in the information standards and a user's Life State.

Referring to FIG. 3A, the information marketplace may consist of categories. An information marketplace transaction may include multiple categories, attributes, and subattributes from a user's life state. A life state may consist of multiple categories 310. It is envisaged that each of the many aspects of a user's life can be mapped to a category of the life state, which may be offered for sale or access in the information marketplace, which is based on information in a user's life state 305. Each of the categories 310 may consist of many attributes 315 and sub-attributes 320. Although not illustrated, each of the sub-attributes 320 may consist of many deeper levels of sub-attributes. For example, under the sub-attribute 1, there may be sub-attribute 1.1, sub-attribute 1.2, etc. The categories, attributes, and/or sub-attributes may be dynamic and may be managed by the user or an administrator. The categories, attributes, and/or sub-attributes may be offered for sale or access according to gatekeeping and security attributes 325. The ability to view the updated categories, the attributes and the sub-attributes illustrate the dynamic nature of the information marketplace offers and transactions, as well as the gatekeeping and security mechanisms that may protect a user's privacy rights and allow consensual access to the user's information.

Security attributes, gatekeeping attributes, information marketplace parameters, and the user's preferences may determine how life state categories, attributes, and sub-attributes are accessed, offered for sale, and transacted. Following are some possible categories, attributes, and sub-attributes. There may be a category “psychological.” Under this category, there may be attributes “personality type”, “planner/spontaneous”, and “conservative/risk taking.” Under the attribute “personality type”, there may be the sub-attribute “Myers Briggs Classification” and the sub-attribute “Functions of Consciousness”, and the sub-attribute “Extraversion/Introversion.” Under the category “personal core information”, there may be several attributes such as “age”, “sex”, “marital status”, “family”, “physical”, “health”, “ethnicity”, etc. Under the attribute “health”, there may be a sub-attribute such as “ailments”, and under the sub-attribute “ailments”, there may be lower level of sub-attributes such as “treatments”, “medication”, etc. A value for the sub-attribute “ailments” may be “diabetes.” A value for the attribute “marital status” may be “single.” As mentioned, the number of categories, attributes and sub-attributes may be large since they together describe the users' life state. There may be security and gatekeeping attribute settings that allow a user to offer for sale all information under a personality type attribute. The user may use other settings to ensure that other psychological attributes, as well as the psychological attribute overall, are not accessible. The user may further use security and gatekeeping attributes to determine parameters that specify how the information is accessible. For example, the user may configure security and gatekeeping attributes for some personality type sub-attributes to be accessible to other organizations or third parties that would like to provide information to the user, while other personality type sub-attributes are configured to allow the information to be accessible only by sale in the information marketplace. Other example categories, attributes, or sub-attributes and their design are included in the corresponding applications and provisional application which is described in the related application section at the top of this document.

As the user's life changes, the values for the user's life state, categories, attributes, sub-attributes may be updated, which may update the information in the user's information marketplace. As the life state, categories, attributes or sub-attributes change, the Information in the information marketplace may expire or be updated, according to information marketplace and/or transaction parameters, price, information attributes, and information sub-attributes. For example, a user may sell information about a medical condition they have. If the medical condition changes, then under some embodiments the sold information may be updated, or the buyer notified that the information has changed, or there may be no notification. The number combinations of categories, attributes, and sub-attributes (and deeper levels of sub-attributes) may be extensive, and may represent a snap shot of the life of a user, which may require information marketplace filtering. It may be noted that this is different from the traditional marketplaces and/or information repositories and portals displayed/used since the information included in a traditional portal or repository is either very limited, not as extensive as what is included in a user's life state, unstructured, incomplete, in violation of a user's privacy rights, and/or not offered for sale or granting. For example, an information directory may offer various information for background checks or other applications. This information rarely has the owner's consent, is not complete, is structured exclusively for a few specific applications that don't target the user's behavior and life state, and are not for sale or granting without specific and limited use. The information marketplace may be configured to provide interview tools, wizards, application program interfaces, or applications that gather and structure the information for sale or granting about the user life state to the relevant life state, attribute and sub-attribute values.

FIG. 3B illustrates an example of an information marketplace 330, in accordance with some embodiments. The information marketplaces 330, lifestates 325 may represent the transactions of many users. The life view 360 may display information regarding the information marketplace. In the current example, the collection of information marketplaces 330 includes life states 325, which includes the life states 350 and 355. The funnel shape associated with each of the life states 350 and 355 illustrates the concept of using a life state to funnel or filter the information based on life state security and/or gatekeeping attributes into the information marketplace 330. The life state helps funneling the information so that only filtered information and information deemed available for sale or granting by the user is available to the information marketplace. In other words, the life state enables the right type of information to become available to other parties interested in buying or obtaining information about the user. A life view 360 may also display custom user information that displays many aspects of the information offered for sale or granting, as well as past transactions, in the user's life view.

The information marketplace may also include notifications that are sent to other parties when a user visits that party's web site or other embodiments. For example, if the user visits a web site that uses entertainment information, yet the web site's organization does not have any entertainment based information from the user, the web site organization may be notified that the user that is visiting the web site has relevant entertainment information in their life state and/or information marketplace.

FIG. 3C illustrates an example of information marketplace offers, bids, and transactions with some embodiments. This example illustrates a life state structure similar to the example described in FIG. 3A. A user has complete control how the access to the user's life state is granted or offered for sale. The granting or sale of the access can be at the category level, the attribute level, the sub-attribute level, or combinations thereof. A combination of one or more categories, one or more attributes, and/or one or more sub-attributes is referred to as an access group. Each access group may be viewed or offered for sale or granting as a subset of the life state. A third party can be granted permission to access or procure various areas of the user's life state The content of an access group may vary and may be changed by the user. As will be described, the granting of the access is performed using the security and gatekeeping features, as well as information marketplace attributes and parameters.

As illustrated, the information marketplace may use bids delivered by third parties 330, 332, 334, 336, and 338, which are in response to category and attribute offers 310, 340, 342, 346, 348, 349. The arrows from the bids to the user's categories 310, attributes 348, and sub-attributes 342 indicate information offered for sale or granting by the user, according to the user's security, gatekeeping, and information marketplace attributes and parameters defined in the life state. The transaction parameters 350 may determine third party or other filters, grouping, display characteristics, frequencies, and other parameters for the user's information transaction 360. The information marketplace may transact information among many users and third parties, as defined by the life state attributes, security, and gatekeeping attributes and information marketplace transaction parameters. The offers may include dynamic information delivered from the third parties, which may include expiration dates where the information is updated through the expiration date. The information marketplace may also include information regarding the delivered information quality and alignment. For example, a user may offer information regarding their preference and behavior in regards to product introductions relating to laptops, and the user information with the highest alignment and quality from the user's life state may be offered for sale at a premium due to the high alignment and quality. Information bids 330 are granted access to the attribute offers 340 which consist of information for sale or granting that is associated with some attributes under the category 1. The information bid 332 is granted access to the sub-attribute offers 342 which consist of some sub-attribute values under the attribute 1 that are offered for sale. The third party 334 is granted access to the sub-attribute offers 346 which consist of some other sub-attributes under the attribute 1 that are offered for sale. The third party 336 is granted access to the attribute offers 348 which consist of some attributes under the category 1. The third party 336 is also granted access to the attribute and sub-attribute offers 346. The third party 338 is granted access to the category and attribute offers 348 which consists of values associated with some attributes under the category 1. The third party 338 is also granted access to the category and attribute offers 349 which consists of values associated with the attributes and sub-attributes under the category 3 and category 4. These offer access and granting permission examples illustrate some of the flexibilities and control that a user may have with the user's information marketplace offers and the third party's bids. The directions of the arrows from the bids to the offers illustrate the granting of the permission by the user to the third parties in the information marketplace.

FIG. 3D illustrates an example diagram of a group of customers or third parties, in accordance with some embodiments. The diagram includes an information marketplace similar to the information marketplace described with FIG. 3B. For some embodiments, a third party may have to be approved before it can be granted permission to participate in the information marketplace, where life state information may be offered for sale or granting to those groups. A set of criteria may be used to evaluate and approve a third party to the life state or information marketplace. The set of criteria may be determined by a management team or an administrator associated with the information delivery system and/or the information marketplace. For example, the criteria may include one or more of the following: whether or not the third party is a participant in the life state system; whether or not, or how, the third party provides information, content, products, or services for the categories/attributes/sub-attributes for the information being offered for sale; qualifications for access through fees, licensing, etc. the quality of the third party when it maps its services and information to the life state structure; the reputation of the third party, the likelihood that the third party will continue to perform as a partner and a leader in the category/attribute/sub-attribute that it is assigned to, etc. Other criteria's may also be used.

A license may be sold or granted to the third party. The license may give the third party permission to copy or use the users' life states for a defined period. There may be an expiration date, and the license may need to be renewed. The license may be revoked if the third party does not follow the terms of the purchase or granting. The third parties may use the copies of the life states according to predefined or dynamic terms, or at their discretion. For example, the third parties may choose to filter its information, or the third parties may customize its web site for the users when the users visit the websites of the third parties. The third parties may obtain specific user identification as part of the transaction, allowing future activities, customization, and interactions concerning the user. Depending on whether or not a third party follows the terms of sale, a license may remain active, suspended, or revoked. Other license status may also be used. Security credentials including user identification (ID) and password may be assigned to an approved third party. A third party who has been approved is referred to herein as a customer. For some embodiments, only those customers whose status is active and have completed a transaction allowing access to a user's information may be granted permission to access the users' life states. There may be different types of customer or user. For example, a customer or user may be an individual, an organization, a governmental agency, etc. The customers may also be defined based on the type of content, services, or products they offer. For example, the type “Search Engines” or “Software” may be used for Microsoft® and Google® when they become customers, and the customer type “Government” may be used when the Medicare office may want to procure the users' life states for benefits administration. For example, a user may offer for sale its age and medical insurance coverage state, which in turn may be procured by any customer with the type “Government”. For some embodiments, a user may be an organization or a collection of users with common attributes. For example, Sony may be a user with their own life state attributes that range from organizational attributes, to current initiatives, supplier bids, etc., which in turn may be offered for sale or granting to other life state customers, and Sony®'s PlayStation division may also be a user with its own attributes that range from divisional or group specific attributes that may in turn be for sale. For example, the user Sony® PlayStation may have an attribute value that specifies when it will allow bids for new games to be developed for a new video game console, which in turn can be offered for sale or granting to other life state customers.

In the current example, the block 324 represents all of the customers. They include the customers 360, 361 and 362. These customers have been granted permission to the information marketplace 370. The information marketplace may access one or multiple life states as defined by the user and embodiments herein and the information marketplace parameters. As described with FIG. 3C, the parameters of each of the transactions 350 may be different. The directions of the arrows from the information marketplace 305 to the information transaction 360 illustrate the bids to procure or obtain information from the users. For some embodiments, customers may indirectly purchase information about a user that is derived from the user's experiences with another third party. For example, Amazon may purchase information from a user (such as the user's preferences and behavior regarding new technology product releases), and this information may have been derived wholly or in part by the user loading information from Amazon® into its life state, where the user's associated life state technology adoption categories, attributes, and sub-attributes are then purchased by another third party such as Apple®.

FIG. 3E illustrates an example diagram of an information marketplace, in accordance with some embodiments. The information marketplace may be configured to display filtered information from the life states 350, 355, 360 on behalf of the users and present that information to the information customers 360, 361, 362. Each user is associated with a life state. In the current example, the life state 351 is associated with the life state information market section 350, the life state 356 is associated with the life state information market section 355, and the life state 361 is associated with the life state information market section 360. To access the information marketplace, the users and customers may need to log in to the information marketplace server using their user IDs and passwords. The information offered for sale or granting, and the information transacted may also be stored as floating variable, cache, memory, etc. on any device or system that has access to the information marketplace, life state, or life view. The directions of the arrows to the information marketplace sections 350, 355 and 360 from the respective life states 351, 356 and 361 illustrates the flow of the filtered information to the information marketplace. The direction of the arrows to the information customers 360, 361, 362 illustrate the flow of the sold or granted information from the information marketplace sections to the information customers. The information available via the life state information marketplace section 350 may be different from the information available via the life state information marketplace section 355 because the life state 351 may be different from the life state 356.

### Server Applications

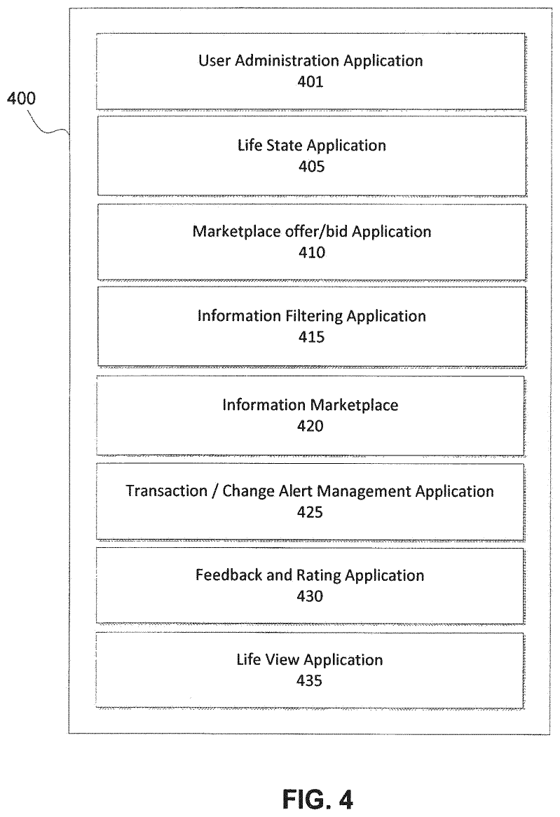

FIG. 4 illustrates example applications that may be used to enable the users and customers to transact the filtered information, in accordance with some embodiments. The information marketplace server 400 may include user administration application 401, life state application 405, market offer/bid application 410, information filtering application 415, information marketplace application 420, transaction/change alert management application 425, feedback and rating application 430 and life view application 435. It may be noted that even though these applications are described herein as separate applications, the functionalities of two or more of them may be combined into one application. These applications may be used to pull filtered information from various resources and allow users to offer the filtered information for sale or granting. The resources may include values entered by the user or Internet websites or information associated with, or entered by, life state partners. Non-Internet resources may also be used. For example, a user's life state information may be replicated, available for sale or granting on devices, appliances, or any means that allows the user to control how to sell or grant the user's life state information to third parties. It may be noted that not every device or appliance used in embodiments of the invention is a filtered information pushing or information marketplace mechanism. That is, some devices may be registered in the core system to receive the pushed information, and then the devices may only function in the ecosystem as a conduit for the pushing of the filtered information to the users or allowing users to offer their information for sale or granting, and allowing customers to purchase or obtain the information.

The user administration application 401 may be configured to manage the users and the customers including setting up their user accounts, user IDs, passwords, etc. These user accounts allow the users and the customers to participate in the information delivery system and marketplace associated with the information marketplace server 400. The user accounts may be stored in the database associated with the information marketplace server 400. The user accounts of the users may be encrypted and stored locally in the client computing systems. The client computing systems may be appliances, devices, etc. This enables a customer to recognize the user and the user's information as part of the information marketplace system when the user visits a website associated with the customer. This may enable the customer to keep track of the user's behavior while visiting that website to determine if the user's behavior is aligned with the user's life state information. The user administration application 401 may also keep track of user type (e.g., user, guest, individual, organization, etc.), customer type (e.g., individual, organization, etc.), customer category (e.g., content provider, advertiser, etc.), customer and user status (e.g., active, revoked, suspended, pending, etc.), the type of license granted to the customer, transaction types (one-time, dynamic, etc.), information marketplace parameters, etc. It may be noted that the user may be an individual, an organization, or a business, etc.

The life state application 405 may be configured to perform operations associated with forming and maintaining of the life states of many users. The life state application 405 may be configured to enable an administrator to define a life state structure including the categories, attributes, and sub-attributes. The life state application 405 may be configured to present a life state interface to allow the users to provide and update the values for the attributes and sub-attributes for their life states. For some embodiments, the life state application 405 may also be configured to perform consistency verification to determine if the user provides inconsistent values for the attributes and/or sub-attributes and to notify the user if inconsistencies are found. Other consistency verification may include user behavior versus the attributes, and one category/attribute versus another category/attribute.

The life states of all of the users and information marketplace transactions may be securely stored in the database associated with the information marketplace server 400 and in a relational structure with tables and fields arranged in ways that are logical and meaningful to the user and customer. When the users update their life states, the updated values may be stored in the same database. It is possible that the updates may also be propagated to the information marketplace and other replicated systems, devices, appliances that locally store the values, as well as third parties that customize their web sites according to these values. This propagation would be similar to a change trigger.

For some embodiments, a copy of the user's life state and parts of the information marketplace may be downloaded and stored in the memory of the client computing system associated with the user or customer. The copy in the client computing system is referred to as a local copy of the life state or a local copy of the information marketplace. It is possible that, in some embodiments, only a portion of the information or only related information may be stored in the client computing system. This is because getting the exact copy may be too big.

Sometimes it might be just the user or customer ID that's stored in floating variables, memory, cache, etc., which then pulls the user's life state and is used in the information marketplace when needed.

Embodiments of the invention may enable the user's life state to be used in a more global information delivery with every conduit that the user may use for information as opposed to strictly web searches. For example, when the user visits a website, the user may have the option of selecting to apply the user's life state with that web site. The option may be a toggle that can be turned on or off, and the information associated with that website may toggle along with this option. If the web site maintains user accounts, the user may connect user ID with the information delivery system with the website. The user ID from the information marketplace may also be available and used to associate the user visiting the website with any information transactions made involving the user. This will allow greater flexibility and efficiency when the website accesses the user's life state, as well as comparing actual user behavior against information procured or obtained regarding a user's information in the information marketplace.

The marketplace offer/bid application 410 may be configured to enable the information marketplace server 400 to communicate with one or more servers of the life state users. For example, this may enable the information marketplace server to access the users' life states as defined by the users' gate keeping, security, and information marketplace server features. This may include interfacing with the users' life views, third party customized websites, filtered search results, etc.

The information filtering application 415 may be configured to share the life states of a user with the information marketplace server, limited to the level of permission granted by the user and the information marketplace parameters. The information filtering application 415 may also be configured to apply the security and the gate keeping features set by the user. As described, the information marketplace server may use the user's life state to offer the user's information for sale or granting. The information filtering application 415 may also present the filtered information to the user via the user's life view.

The information marketplace application 420 may be configured to allow the users to define how the information provided to the users may be offered for sale or granting, and transacted, according to the embodiments herein. The information marketplace application 420 may be configured to provide an internet based commerce platform that allows the customers (third parties) to buy user information from sellers (users). The commerce platform may include bid and offer parameters and transactions. The layout interface may include parameters to control the fields and dimensions of the layout. The user may define multiple methods of offering their information defined by the user and the user's life state. For example, the user may define parameters that cause their information in regards to music entertainment preferences, history, and purchases to be offered for sale; the user may also offer all entertainment preferences, history, and purchases to be offered for sale as a license, which may allow customer access to changes to this information over a specified timeframe. As mentioned, each of the users may be associated with a life view. The user accesses the life view by logging into the information delivery system using the assigned user ID and password. The life view may display information regarding the user's activity, bids/offers, and transactions in the information marketplace application 420.

The transaction/change alert management application 425 may be configured to notify the customers whenever there is a change that may affect the transacted information or the process of providing the transacted information to the customers. The changes originate from the users or life state third parties, in the user's life state and may also be reflected in the user's life view. The change may be necessary to address a critical update in the user's life state information, which is also relevant to the terms of the transaction. For example, the customer may have purchased the user's information that relates to electronic based assets, such as a Samsung® Galaxy S4; whereas the user's life state gets updated by disposing of the Samsung® Galaxy S4 and replacing the user's ownership of an iPad Air device. It is important to note that such updates may be factored into an information transaction. There may also be changes originating internally by a management team associated with the information delivery system to enhance the information filtering services for the customers and/or the users. For another example, it may be necessary to add a new category, to migrate an attribute from one category to another category, to delete an attribute, to change the meaning of an attribute, etc., and these changes may be propagated to customers' transactions that include transaction terms for this propagation. These changes are sourced in the user's life state and may also be reflected in the user's life view. Changes may also originate externally by others. For example, a user may have configured their life state to automatically update one of their attributes, such as their asset inventory, upon each transaction at Amazon.com. The transaction/change alert management application 425 may also be configured to notify a customer when the user modifies the user's life state. Similarly, the transaction/change alert management application 425 may notify a user or customer when the information is modified, where the customer is only notified of the information change but may be required to enter a new transaction to receive the specific information change.

The rating application 430 is configured to improve the quality of the transacted information. This includes providing a mechanism to allow the life state partners to rate the users' behavior as a measurement of accuracy of the user's life state information. The ratings are based on how accurately the information in the user's life state is aligned with the user's actual behavior, as well as how accurate this alignment is in comparison of life state behavioral alignment of other users. These ratings may be used by users in pricing the information they offer for sale, as well as by the customers when valuing the information offered for sale. Information offered for sale with a higher accuracy may justify a higher value of the information offered. For example, if a user has a high alignment, as a rating of their behavior aligning with their life state information, that information may be more valuable and justify a higher price that a user offering the same information that has low alignment. Following are some conceptual examples of the ratings for a user's behavioral alignment, and these ratings may take any other form as well, such as a percentage, or other rating scale:
None—The user's behavior is not aligned with information from the user's life state
Low—The user's behavior is minimally aligned with the user's life state
Moderate—The user's behavior is mostly aligned with the user's life state
High—The user's behavior is significantly aligned with the user's life state

In order for a user to maintain accurate information, it may be necessary for the user to keep the user's life state updated or utilize automatic loader programs that accomplish the same. If the user's behavior remains non-aligned with the user's life state for an extended period of time, the information provided for sale or granting by the user may carry little value in the marketplace. A customer may also rate how accurate a user's behavior is following the procurement or obtaining of that user's information. These ratings may be available in the information marketplace. For example, through interactions with a customer, an agent of the customer, or in behavior that is visible to the customer, or via other means, a customer may rate the user's behavior following a transaction as being highly aligned with the information transacted; this allows the value of the information transacted to be higher; and information transacted that is not followed by aligned behavior may be valued lower. Following are some conceptual examples of the ratings for a user, and these ratings may take any other form as well, such as a percentage, or any other rating scale:
None—The user's transactions are not aligned with the user's transacted information
Low—The user's transactions are minimally aligned with the user's transacted information
Moderate—The user's transactions are mostly aligned with the user's transacted information
High—The user's transactions are significantly aligned with the user's transacted information

The lifeview application 435 may be configured to implement a custom view or portal where users may view custom information about their life state in a life view. In some embodiments, the marketplace information may be displayed in this life view. The information in the life view application displays information according to the information marketplace parameters, and security, and gatekeeping attributes. For some embodiments, the life view may display current information offers, bids, and other transaction information from the information marketplace.

### Security and Gatekeeping Features

The information marketplace may access the life states of the user according to the user's life state security and gatekeeping features, and the information marketplace parameters. Referring to FIG. 3D, users may use different security, gatekeeping, and information marketplace features 350, 355, 360 to control how the information is offered for sale or granting. The security, gatekeeping, and information marketplace features 325 control the visibility at the global level, category level, attribute level or sub-attribute levels. The features 325 set at the global level apply to all categories, attributes, and sub-attributes. The features 325 set at the category level apply to all corresponding attributes and sub-attributes. The features 325 set at the attribute level apply to all corresponding sub-attributes and so on. When there is a difference among the settings of these features, the settings of the features at the lower level takes precedence over the settings of the features at the corresponding higher level. For example, if a user sets a value at the global level, and then sets another value at the category level, the value at the category level will determine the setting of the features for that category level, but only for that category level. Following are some examples settings of the marketplace security, gatekeeping, and parameter features 325:
Not available—No information relating to the category, attribute, or sub-attribute is available to any customer or any other parties;
Available for sale only to preferred/approved list—No information relating to the category, attribute, or sub-attribute is available for sale to any customer or other parties unless they are included in the user's list of approved or preferred customers and/or parties;
Available for sale—Information relating to the category, attribute, or sub-attribute is available to any customer or third party;
Available for sale by industry category—No information relating to the category, attribute, or sub-attribute is available for sale to any customer or other parties unless the customer or other party is a member of a designated industry; and
Additional feature settings may exist for time frames, different types of transactions, customer ratings, and other aspects that relate to offering the user's information for sale or granting. For one example, a type may exist that allows customers to offer a user a discount on products or services in exchange for the user's information.

For some embodiments, there may also be transaction types that grant a customer or third party additional gatekeeping features, such as frequency and precedence controls that will affect how the system queues up information from that customer in relation to other parties for the user's life state and life view.

For some embodiments, the settings of the marketplace features may be subordinate to the settings of the security features. The settings of the security features for a given category or attribute may override the settings of the marketplace features for the same category or attribute. For example, if the user sets a security feature for a category as “Not available”, but the user sets a marketplace feature for an attribute in that category as “Not available”, then the information marketplace may ignore the setting at the attribute level. This means that the values of the attributes and sub-attributes associated with that category may not be available to any of the partners. The “Not available” security setting may be interpreted as a blocking setting which blocks any cascading information availability related to a category, attribute, or sub-attribute. In this example, the information marketplace may not have access to any information related to the “Not available” category even though the attribute level feature for that category is set as “Available.” In other embodiments, a setting at the category level of “Determined at the attribute level” or “Determined at the sub-attribute level(s)” may allow a clear interpretation of the setting that the control is determined at a lower level than at the category level. The combination of the security and marketplace features allow the users to highly customize how the user grants permission to the user's information in the information marketplace in terms of depth and granularity of information available, types of use, types of transactions, types of customers that can transact, etc. In some embodiments, the security, gatekeeping, and information market attributes and settings may be programed to automatically change based on a change. For example, changing a global setting to not allow any information be to available may automatically change all downstream attributes and settings to synchronize the lower level settings to this change.

In general, marketplace features are different from security in that they determine how information is transacted in the user's information marketplace. This includes controlling the types of sale/granting, transaction types, frequency of informational updates, extent of information for sale/granting, type of information for sale/granting, and volume of transacted information that is offered for sale/granting by the user and purchased or obtained by the customer. This is where the information marketplace implementation becomes important. Under some embodiments, a user may want to allow information for a specific attribute to be sold in the marketplace, where no information is delivered for that attribute to the user's life state application, then the security settings would allow visibility in the marketplace, and the other life state gatekeeping settings would not allow information delivery to the life state or portal (or when surfing to external sites).

### Automatic Populating and Updating Attribute Values

For some embodiments, the life state application 405 described with FIG. 4 may be configured to auto-populate the attributes and sub-attributes with values. The values may be derived from the life state partners. It is possible that the partners may accumulate extensive information about a user's behavior, which in turn gives them the ability to populate the users attribute values according to this behavior. For example, a set of attributes for a user's asset inventory may include various products, etc. Amazon® could easily populate the asset inventory according to the user's purchase history. A user's behavior may also be accumulated by the user's life view, by tracking which links or other interactions a user makes within their life view. There may be an option to allow the user to select the auto-populating feature. The information marketplace may allow the user to transact this information with customers and third parties.

For some embodiments, the life state application 405 described with FIG. 4 may be configured to automatically update the life state of a user. There may be a setting that enables the user to indicate that the automatic update of the user's life state is allowed. The updates may be based on transactions that the user engages in or behavior the user engages in while using their life view, and may be performed periodically. It is possible that user's behavior may not be consistent with the user's life state. For example, the user indicates in the life state category and attribute values that would lead to avoidance of water sports related transaction behavior. However, through the user's life view, the user frequently views websites that relate to water sports. In this scenario, the life state application 405 may modify the user's life state to align with the user's behavior. This may include automatically translating a user's life view behavior and interaction into values for the attributes and/or sub-attributes. The user can also review, assess, and override any values that are set from an auto update, if the manual setting of the values better reflects the user's life state. This ensures that the user's life state is up to date. These embodiments may allow the user to offer these types of updates to information transacted in the information marketplace, depending on the transaction parameters.

Network with Partner Servers

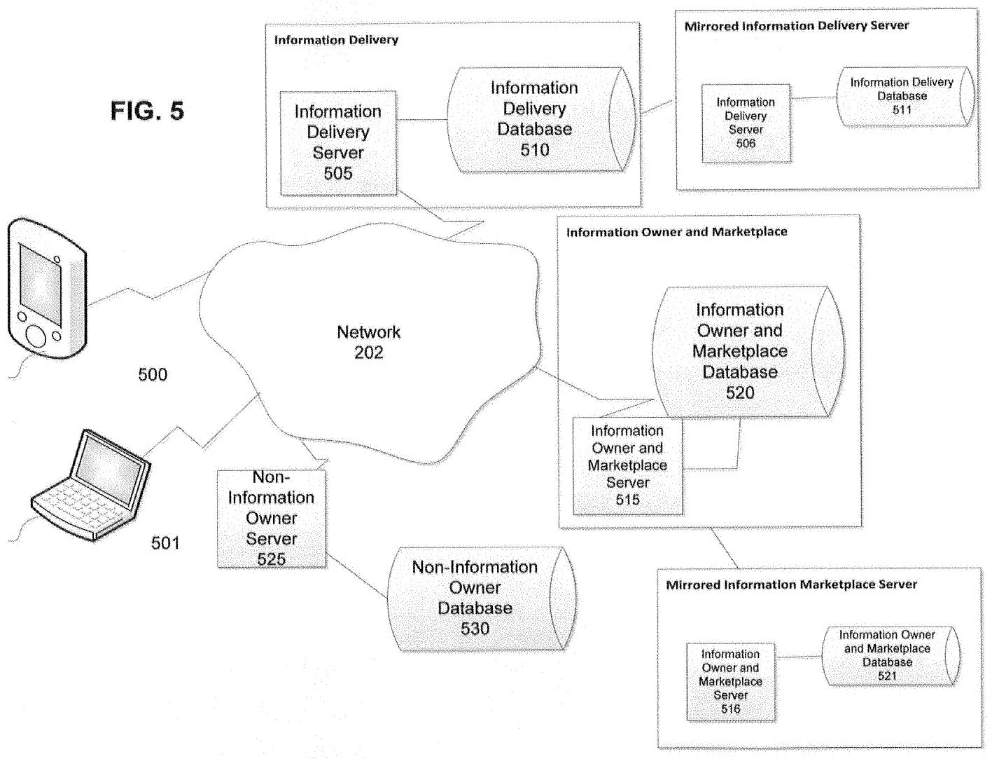

FIG. 5 illustrates an example of a network with an information delivery and marketplace server, in accordance with some embodiments. The network may include an information delivery server 505 coupled with database 510 and connected to network 202. Also connected to the network 202 are client computing systems 500 and 501, partner servers 515 and 525 and their respective databases 520 and 530. The client computing systems 500, 501 may be configured with browser software or other means to access the services of the information delivery server 505 and the information owner and marketplace server using the Internet. The client computing systems 500, 501 may be connected to the network 202 via wired or wireless communication (e.g., Wi-Fi, RF, Bluetooth, etc.). For example, a user may use the client computing system 500 to register with the information delivery and/or information marketplace servers 505 and 515, input values to configure the user's life state and information marketplace, set up security features and gatekeeping features, and set up marketplace parameters. The user may use the client computing system 500 to log in to the information delivery server 505 and access the user's life state to view the filtered information. The user may use the client computing system 500 to also log in to the information marketplace server 515 and access the user's information marketplace. The user's life state, security features, gatekeeping features, and filtered information may be stored in the database 510. The user's information marketplace, security features, gatekeeping features, and marketplace parameters may be stored in the database 520. The network infrastructure described in FIG. 5 may also be used to enable the partners as information marketplace customers to participate in the marketplace to procure the life state information.

Embodiments of the invention may also include mobile-enabled implementations to enable the users to user the mobile devices to communicate with the information delivery and information marketplace servers. As illustrated in FIG. 5, the mobile device 500 may be used by a user to connect to the information delivery and/or information marketplace server 505 via the network 202. Having the mobile capability may enable the users to provide updates and make information transactions on an impulse basis, especially at a time when the user is experiencing something that may need to be reflected in the user's life state, or that has information marketplace value due to the young age of the information. The information delivery server 505 may enable the user to download a mobile application for these purposes. The mobile application may include user interfaces that make it easier to provide information about the user's experience, as well as transact information relating to those experiences or in general. For example, the user may be watching a movie, and the user may use the mobile device to indicate the name of the movie, how much the user enjoys watching that movie, whether or not the user may be interested in a sequel, which in turn may also be available for sale at that time. That action will be analyzed by the information delivery server, the user's entertainment attribute may be updated, and the information transaction process may begin and complete. These embodiments may also be immediately available and reflected in the user's life view.

### Mirroring and Distributed Processing

Referring to FIG. 5, in some embodiments, there may be one or more mirrored servers 506 configured to perform tasks similar to the information delivery servers 505 to accommodate the demand and usage by many users; and there may be one or more mirrored servers 516 configured to perform tasks similar to the information owner and marketplace servers 515 to accommodate the demand and usage by many users. These mirrored servers may be located at different geographical areas. For example, at a first site, there may be one or more mirrored servers 506 and/or 516. Each of the mirrored servers 506 or 516 may be associated with a mirrored database 511 or 521 which may be configured to store information similar to the databases 510 and/or 520. Further, there may be multiple mirrored sites, each having similar hardware and software configuration and set up as the first site. The multiple sites may collaborate with one another in a distributed manner to help speed up the process enabling the users to set up, maintain, and transact their life states, interacting with the partner and third party servers, receiving the filtered information from the partners, transacting their information with third parties, enabling the users to configure their life views, presenting the filtered information to the users' life views, etc. Using mirrored servers and distributed processing may help the users to view most relevant and accurate filtered information in their information marketplaces and life views in near real time.

It should be noted that there are many different distributed processing architectures and the description provided here is not meant to limit to a particular architecture, but rather to convey that distributed processing may be implemented to provide better user experience for some embodiments.

### Flow Diagrams

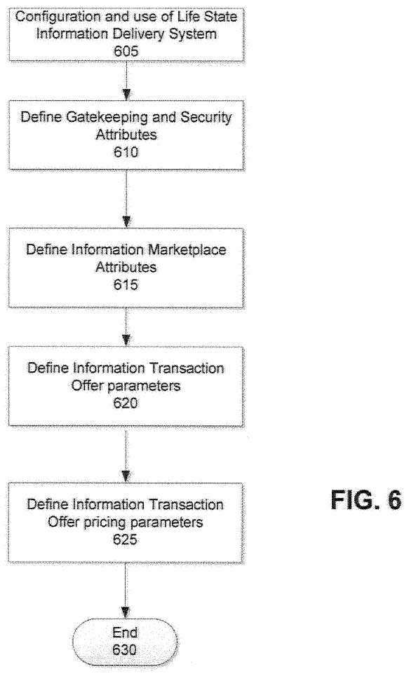

FIGS. 6-11 describe various processes that may be performed by the information delivery system and information marketplace. Each process includes multiple blocks, with each block representing one or more operations. The sequences of the blocks may vary depending on the implementations. Some operations may be combined while some operations may be separated into multiple operations.

FIG. 6 illustrates an example flow diagram of a process that may be performed by an information delivery system to set up the information marketplace, in accordance with some embodiments. The process is associated with operations that may be performed by the information delivery server 505 and information owner and marketplace server 515 described with FIG. 5. At block 605, the structure of a life state is defined. This may include defining the categories, the attributes, the sub-attributes, and any deeper level of sub-attributes. For example, each of the attributes, and sub-attributes may be represented by fields in a database table with each of the fields having a value or as programming variables in RAM or cache, or as other embodiments. Each time a new user is registered with the information delivery server 505 and information owner and marketplace server 515, life state and information marketplace gatekeeping and security attributes may be associated with and configured by that user, as shown in block 610. The user may configure how the user's life state information interacts with the information marketplace by the associated information marketplace attributes for that user's information, as shown in block 615. The user and third parties may define information transaction offer parameters, which may determine various aspects of the information offered for purchase or granting by the user or a third party, as shown in block 620. The user or third party may define information transaction offer pricing parameters, that determine pricing values and parameters for the information transaction offer, as shown in block 625.

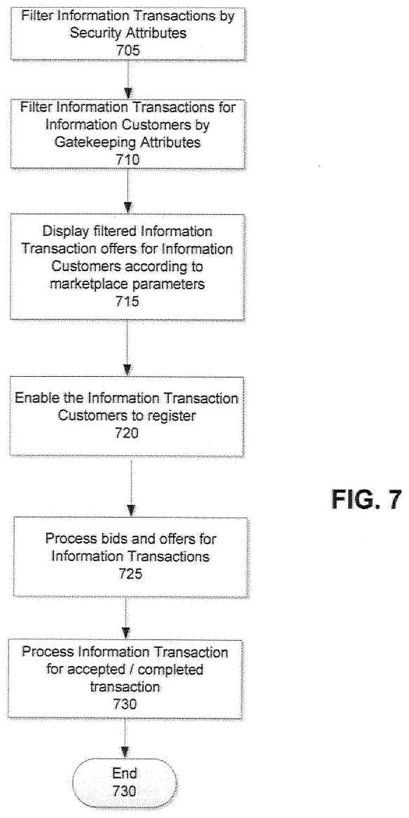

FIG. 7 illustrates an example flow diagram of a process that may be performed by an information delivery system to allow the users to present the filtered information for sale or granting, in accordance with some embodiments. The process is associated with operations that may be performed by the information delivery server 505 and the Information Owner and Marketplace Server 515 described with FIG. 5. A user's security attributes 705 determine overall access to the user's filtered information. A user's gatekeeping attributes 710 determine specific methods of access for the user's filtered information, which may be offered for sale, renting, granting, or other means of transacting. It should also be noted that the user's security and gatekeeping attributes may also determine accessibility for partners in the user's life state to provide the user with customized information. Additional attributes and parameters 715 may be used to further define how the user's information may be offered, purchased, and owned in the information marketplace. For example, a user may sell non-changing information such as birth date, which may be denoted by a marketplace parameter; and the user may also offer a dynamically updating attribute such as his/her favorite music group, which may be enhanced further with an expiration date, after which the customer may no longer receive the updated information. Customers may be registered in this marketplace by a registration process 720. A customer may also be registered as a guest, and other automated methods of registering the customer may also be used from the information delivery system. It is also possible that a customer also exists in the information marketplace as a user, offering information for granting or sale. The user's filtered information is then offered for sale or granting, and in return customers may bid, procure, or obtain the user's filtered information 725. The completed transactions, expirations, and other information, user, and customer based information marketplace parameters may be entered and stored in a transaction record or set of records 730.

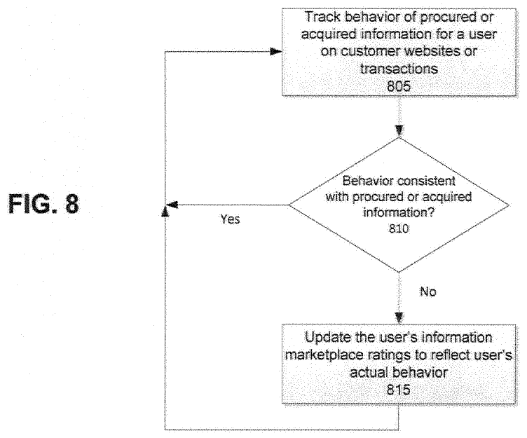

FIG. 8 illustrates an example flow diagram of a process that may track the user's behavior when the user visits a website of the customer or transacts or interacts with a customer. When the user visits the website of a customer or transacts or interacts with the customer, the customer may recognize that the user is a member of the same information delivery system and information marketplace that the customer is associated with; which may allow the customer to track the user's behavior and compare it to the information purchased or obtained by the user. The account of the user and the account of the customer with the information delivery system and the information marketplace may be related or connected. The recognition may be based on the information stored locally on the client computing system of the user or the customer. Alternatively, the user id or customer id may be locally stored or stored in temporary internet/buffer files by associating with another user or customer login, and then the attribute values from the user's life state and transacted information may be obtained, refreshed, or updated.

The tracking operation may include interacting with the customer's server and receiving the tracking information from the customer's server, as shown in block 805. At block 810, a test is performed to determine if the user's behavior is aligned with the procured or obtained user's information. If the user's behavior is aligned, the process flow from block 810 back to block 805 and the determination may continue. However, from the block 810, if the user's behavior is not aligned, the process flow to block 815, where the user's information marketplace ratings may be automatically updated to reflect the user's behavior. From the block 815, the process may flow back to block 805 and the determination may continue. For some embodiments, this process may be performed periodically. An automatic update option may be selected by the user or customer. In this process, the users and customers may also be provided with the attribute values that are not aligned, which may help the user migrate towards better alignment, and also may help the customer understand the user's behavior and perceptions more.

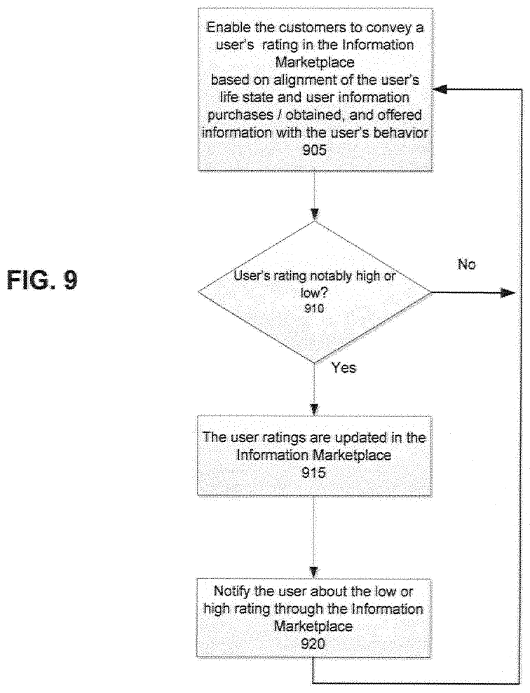

FIG. 9 illustrates an example flow diagram of a process used to rate a user, in accordance with some embodiments. At block 905, the customers are enabled to provide rating information for a user. As described above, a user's life state involves many aspects of the user's life, and it is expected to change as the user experiences changes in the user's life. The changes may be reflected in the user's behavior when, for example, the user visits a website of a customer. The customer may track the user's behaviors, compare those behaviors with the user's life state, compare those behaviors with user information offered, purchased or obtained, and give the user a rating. At block 910, a test is performed to determine the rating for the user. A rating scale may be determined by the management team, and the scale may be the same or different from the scale used to rate a customer. If the rating falls below or rises above defined thresholds, the process may flow to block 1015, where the information delivery system may perform operations to notify the user of the changes in their rating. For example, warnings may be sent to the user to notify the user of the low rating; requests may be sent to the user to notify the user to update the user's life state or information offered for sale or granting; the status of the user may be changed, etc. as shown in block 920. From the block 910, if the rating is not notable as a high or low rating, the process may flow back to block 905 to collect more ratings. It is possible that the rating of the user may be evaluated after collecting a large collection of rating from many customers. From the block 920, the process may also flow back to the block 905. The user rating operations may be performed periodically or as needed. Embodiments of the invention may merely notify the users of the or high low rating but may not cause any impact on information marketplace offers, bids, or transactions. As such the operations described in block 915 may not be performed. The information marketplace may use any combination of publishing the user ratings or managing them confidentially depending on use and configuration, and as decided by a management team.

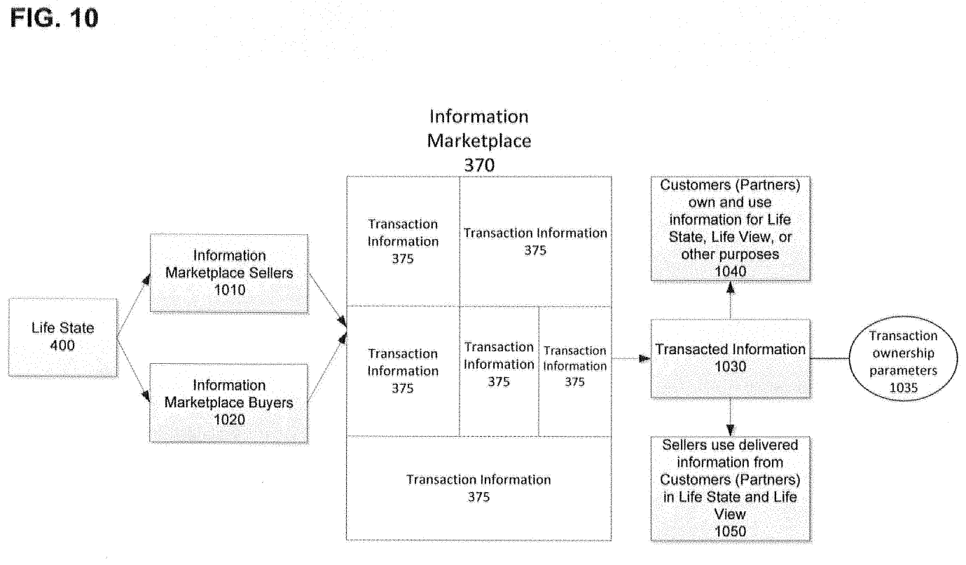

FIG. 10 illustrates an example flow diagram of a process that may be used to enable transacting the user's life state, in accordance with some embodiments. It may be noted that the operations described in the different blocks of this process may not necessarily need to be performed in sequence and some may be performed by different partners. The blocks are meant to convey different ways that the user's life state may be transacted by the customers in various situations.

At block 400, a user's life state information may be offered for sale or granting 1010, and may be procured or obtained by a customer 1020. Upon completing information marketplace transactions 375, the transacted information may be leased, rented, or owned by the customer according to information transaction ownership parameters 1035. Information transaction parameters may also specify other privacy and security attributes. For example, a user may specify that his age is to remain confidential and privileged between the user and the customer, with the exception of other organizations in the entertainment industry; in another example, a user may allow the customer to use the transacted information at their own discretion, which may include selling the information to third parties. Customers may use the information 1040 to interact with a user's life state, life view, or for other purposes according to the transaction parameters. Users may use information that is delivered to his or her life state or life view 1050, which is the result in part or completely from the transacted information 1030.

From the descriptions above, embodiments of the inventions may include a method for transacting information about and relevant to a user's life from a user who is a registered member of an information delivery system, or a user that maintains such information that may be transacted or granted to customers. The transacted information about and relevant to the user's life includes at least (a) demographic information, health information (b), (c) ethnic information, (d) social information, and (e) psychological information. The method also includes enabling the user to grant permissions to a user to offer the information about and relevant to the user's life, wherein the customer is also a registered member of the information delivery system, and wherein the user grants the permission to the customer by transacting information about and relevant to the user's life with the customer and by controlling updates, security, expiration, frequency, extent, types, and volume of filtered information that is purchased or obtained by the customer. In addition, the method includes transacting the filtered information with the customer, wherein the filtered information is used by the customer based on the transaction parameters from the user's life state and the information marketplace parameters, wherein the information about and relevant to the user's life is transacted by the user and the customer using a client computing system associated with the information delivery system and the information marketplace, and wherein the information transactions regarding and relevant to the user's life is received by a server computing system associated with the information delivery system and the information marketplace. Embodiments of the invention also include a computer-readable media that includes instructions that perform the operations similar to the method described above.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. For example, specific examples are provided for shapes and materials; however, embodiments include those variations obvious to a person skilled in the art, such as changing a shape or combining materials together. Further, while some specific embodiments of the invention have been shown the invention is not to be limited to these embodiments. For example, several specific modules have been shown. Each module performs a few specific functions. However, all of these functions could be grouped into one module or even broken down further into scores of modules. Most functions performed by electronic hardware components may be duplicated by software emulation and vice versa. The invention is to be understood as not limited by the specific embodiments described herein, but only by scope of the appended claims.

1.-20. (canceled)

21. A computer-implemented method, comprising:
offering for sale, granting, renting, or leasing, information about and relevant to a user's life from a user who is a registered member of an information delivery system or an information marketplace;
enabling the user to select a customer to sell or grant the information to in terms of information details, duration, expiration, frequency, extent, type, and volume of filtered information that is purchased or obtained by the customer; and
enabling the customer to procure or obtain access to the information.

22. The method of claim 21, wherein the customer is a registered member of the information delivery system or information marketplace.

23. The method of claim 21, wherein the information about and relevant to the user's life includes at least (a) demographic information, (b) health information, (c) ethnic information, (d) social information, and (e) psychological information.

24. The method of claim 23, wherein the information about and relevant to the user's life is provided or sold by the user using a client computing system associated with the information delivery system, information marketplace, and life view.

25. The method of claim 21, further comprising providing a transaction to allow access and updates to filtered information comprising a subset of the information about and relevant to the user's life, wherein the information may be independently configurable based on a life state associated with the user.

26. The method of claim 21, further comprising enabling the user to prioritize, position, and display information regarding marketplace interests, bids, offers, and transactions.

27. The method of claim 21, further comprising establishing a data structure to enable the user to provide and offer for sale or grant the information about and relevant to the user's life.

28. The method of claim 27, wherein establishing the data structure includes establishing fields related to one another, wherein each of the fields is associated with a value and the data structure includes multiple levels such that a field at a lower level is to provide more detailed information and value than a corresponding field at a higher level.

29. The method of claim 28, further comprising defining parameters and settings involving how the information is offered, to what extent, length of time available, extent of updating the information after the transaction, and types of customers that may purchase or obtain the user's information.

30. The method of claim 29, further comprising enabling the user and the customer to complete transactions including the user's information and updating the user's information by using a mobile computing system and a mobile application associated with the information delivery system and life view.

31. The method of claim 21, further comprising enabling the customer to rate the user based on whether the purchased or obtained information from the user is aligned with the user's behavior; and
enabling the user to rate the customer based on whether the information is aligned with behavior of the user when the user visits a website associated with the customer or interacts or transacts with the customer or any of the customer's resources, systems, or personnel.

32. The method of claim 21, further comprising storing transacted information in a secured relational database associated with the information marketplace, information delivery system, and life view; and
enabling the transacted information to be accessed by the customer and user when the user visits a web site of the customer or interacts with the customer, the customer's resources, systems, or personnel.

33. The method of claim 32, wherein customized web pages, systems, databases, and devices are presented to the user when the user interacts with the customer, and wherein the customized depictions are generated by the customer based on the information purchased or obtained about and relevant to the user's life.

34. The method of claim 33, wherein filtered information is presented to the user when the user interacts with the systems, resources, or personnel of the customer, and wherein such information is generated by the customer based on parameters according to the transaction, the user, and based on the information about and relevant to the user's life.

35. A computer-readable media that stores instructions, which when executed by a machine, cause the machine to perform operations comprising:
purchasing or obtaining information about a user's life from a user who is a registered member of an information marketplace;
enabling the user to set parameters to control (a) selling or granting of the information by a customer and (b) volume, frequency, parameters, and transaction types regarding filtered information to be received by the customer from the user;
receiving transacted information from the user, the transacted information being generated by responses to offers from the user for the information or offers to buy or obtain information from the user; and
transacting the filtered information from the user to the customer.

36. The computer-readable media of clam 35, wherein the user and the customer further set the parameters to control (a) type and extent of transacted information, (b) parameters that determine how the information may be managed or used and accessed by the customer following the transaction, (c) the transaction consideration required to complete the transaction, and (d) transactional information such as customer billing information, user information, payment information.

37. The computer-readable media of clam 35, further performing operations comprising: establishing a data structure to enable the user to provide the information, the data structure including fields related to one another, wherein each of the fields is associated with a value representing one aspect of the user's life; automatically populating the fields with a set of baseline values; enabling the user to manually update the baseline values or non-baseline values to more accurately reflect the user's life; enabling the user to rate the customer based on whether the filtered information pushed by the customer is aligned with the information; and enabling the customer to rate the user based on whether the information is aligned with behavior of the user when the user visits a website associated with the customer.

38. The computer-readable media of clam 35, further performing operations comprising: providing a marketplace for the customers, users, or other parties to purchase the information about the user's life; enabling the customer to use the information to customize web pages, systems, and interactions of customer resources or personnel of the customer when the user interacts or transacts with a customer; enabling marketplace information to be displayed in a user life view, according to priority, positioning, and custom display settings; and enabling the user to interact with and use marketplace information in the user life view.

39. The computer-readable media of clam 38, wherein the marketplace is an auction-based marketplace; and wherein the customized web pages, systems, databases, resources, and personnel include content that presents filtered information when that web page, system, database, resource, or personnel are associated with the filtered information.

40. The computer-readable media of clam 38, further performing operations comprising: enabling the user to cause the values of the information transaction fields of the data structure to be updated using a mobile application associated with the information marketplace, the mobile application being configured to enable the user to control and manage transactions and updates that relate to the information marketplace.

41. A system comprising information delivery and marketplace servers connected to a network and configured to: (a) enable a user to sell or grant information about the user's life using a client computing system coupled with the information marketplace servers; (b) enable the user and a customer to set parameters that control transactions including the information by volume, static or dynamic nature, type, or duration of information to be sold or granted to the customer from the user; (c) enable the customer to rate the user based on whether behavior of the user when the user visits a web site, system, database, resource, or personnel of the customer is aligned with the information about the user's life; and (d) enable the user to rate the customer based on whether the information received from the customer is aligned with the information about the user's life.

42. The system of claim 41, wherein the information marketplace servers are further configured to (a) provide a marketplace to enable the customer to purchase the information and (b) update a life view to present the information received from the customer to the user according to priorities, positioning settings, and other user defined settings.

43. The system of claim 41, wherein the information marketplace servers are further configured to (a) receive updated information about the user's life transactions from the user and customer via a mobile computing device, (b) enable the customer to access the information about the user's life to customize the customer's websites, systems, databases, resources, or personnel to integrate the information about the user's life and enable the customer to receive user information transaction updates from the user's life state, and (d) enable the user to access, update, and interact with a user life view via a mobile computing device.
